(19) United States
(12) Patent Application Publication
(10) Pub. No.: US 2024/0271160 A1
(43) Pub. Date: Aug. 15, 2024

(54) PRODUCTION OF ADENO-ASSOCIATED VIRUS VECTOR IN INSECT CELLS

(71) Applicant: Pfizer Inc., New York, NY (US)

(72) Inventors: Erik Sean BARTON, Durham, NC (US); Guogang DONG, Winchester, MA (US); Paul Alexander THODAY, Sterling, MA (US)

(21) Appl. No.: 18/569,368

(22) PCT Filed: Jun. 20, 2022
(86) PCT No.: PCT/IB2022/055715

§ 371 Date: Dec. 12, 2023

Related U.S. Application Data
(60) Provisional application No. 63/364,296, filed on May 6, 2022.
(60) Provisional application No. 63/213,346, filed on Jun. 22, 2021.

(51) Int. Cl. C12N 15/86

(52) U.S. Cl. CPC C12N 15/86; C12N 2710/14044; C12N 2750/14143; C12N 2750/14152

(57) ABSTRACT

The present disclosure relates to compositions and methods for the optimal large-scale production of rAAV vectors using the baculovirus expression vector system in insect cells.

### REFERENCE TO A SEQUENCE LISTING

The instant application contains a Sequence Listing which has been submitted electronically in ASCII format and is hereby incorporate by reference in its entirety. Said ASCII copy, created on May 5, 2022, is named PC072652A PCT_Sequence_Listing_ST25.txt and is 59,346 bytes in size.

### FIELD OF THE INVENTION

The present disclosure relates to optimizing the production of recombinant adeno-associated virus (rAAV) vectors in insect cells. In particular, the present disclosure provides compositions and methods for producing rAAV vectors with improvements in the integrity of viral capsid (cap) proteins and a concomitant increase in the potency of the produced rAAV vectors.

### BACKGROUND OFF THE INVENTION

Recombinant adeno-associated virus (rAAV) vectors are the leading platform for gene therapy delivery due to their low pathogenic potential and their ability to infect both dividing and non-dividing cells. Most commonly, rAAV vectors are produced in mammalian cells (e.g., HEK293 cells, COS cells, HeLa cells). However, large-scale manufacturing of rAAV vectors in mammalian cells remains a challenge and is a limiting factor in the full-scale implementation of rAAV vectors for clinical uses in humans.

rAAV vector production systems based on the ability of baculoviruses to infect insect cells have also been developed (Urabe et al. 2002; Hum. Gene Ther. 13: 1935-1943; Kotin et al. US 2003/0148506; Kotin et al. US 2004/0197895 and Kohlbrenner US 2006/0166363). For example, an insect cell is infected with three different recombinant baculoviruses—one producing the AAV replicase (REP) proteins, a second providing the cap functions for producing the AAV viral structural proteins (VP1, VP2 and VP3) and a third baculovirus comprising a transgene of interest. This baculovirus expression vector (BEV) system can produce large amounts of rAAV vectors in insect cells. However, the effect of baculovirus infection on the potency of resultant rAAV vectors has not been examined in detail. Studies have shown that baculoviral cathepsin (v-CATH) is active on several AAV serotypes, leading to a partial degradation of AAV cap proteins, VP1 and VP2, and a concomitant decrease in rAAV infectivity (Galibert L, Savy A, Dickx Y, Bonnin D, Bertin B, Mushimiyimana I, et al. (2018) Origins of truncated supplementary capsid proteins in rAAV8 vectors produced with the baculovirus system. PLoS ONE 13(11): e0207414).

In view of the above serious limitations of the BEV system, there remains a need to develop efficient and improved methods that allow the production of large amounts of potent rAAV particles.

### SUMMARY OF THE INVENTION

Disclosed and exemplified herein are compositions and methods for the optimal large-scale production of rAAV vectors using the baculovirus expression vector system in insect cells. Those skilled in the art will recognize or be able to ascertain using no more than routine experimentation, many equivalents to the specific embodiments of the invention described herein. Such equivalents are intended to be encompassed by the following embodiments (E).
E1. A method for producing recombinant adeno-associated virus (rAAV) vector comprising:
   contacting an insect cell with one or more recombinant baculovirus(es), each baculovirus comprising a heterologous sequence; and
   culturing the insect cell under suitable conditions and for a time sufficient to produce rAAV vector with no more than 15% clipping between VP1 amino acid residues that correspond to Gly115 and Arg116 of wild-type AAV6 or the corresponding amino acids in the VP1 protein of another AAV serotype.
E2. A method for producing recombinant adeno-associated virus (rAAV) vector comprising:
   contacting an insect cell with one or more recombinant baculovirus(es), each baculovirus comprising a heterologous sequence; and
   culturing the insect cell under suitable conditions and for a time sufficient to produce rAAV vector with no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype.
E3. A method for increasing in vitro potency of a recombinant adeno-associated viral (AAV) vector comprising:
   contacting an insect cell with one or more recombinant baculovirus(es), each baculovirus comprising a heterologous sequence; and
   optimizing the time for culturing the insect cell under suitable conditions such that the in vitropotency of the rAAV vector is between 10%-500% as compared to a reference standard with no more than 15% clipping between VP1 amino acid residues that correspond to Gly115 and Arg116 of wild-type AAV6 or the corresponding amino acids in the VP1 protein of another AAV serotype.
E4. A method for increasing in vitro potency of a recombinant adeno-associated viral (AAV) vector comprising:
   contacting an insect cell with one or more recombinant baculovirus(es), each baculovirus comprising a heterologous sequence; and
   optimizing the time for culturing the insect cell under suitable conditions such that the in vitro potency of the rAAV vector is between 10%-500% as compared to a reference standard with no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype.
E5. The method of any one of E1-E4, wherein the insect cells are cultured for a time sufficient to produce rAAV vector with no more than 75% clipping between VP1 and VP2 amino acid residues that correspond to Gly189 and Glu190 and between VP1 amino acid residues that correspond to Gly115 and Arg116 of wild-type AAV6, or the corresponding amino acids in the VP1 and VP2 proteins of another AAV serotype.
E6. The method of any one of E1-E5, wherein the rAAV vector has an in vitro potency of at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard.
E7. The method of E6, wherein the in vitro potency is measured using a colorimetric assay, a chromogenic assay, an ELISA-based assay, quantitative PCR, and/or Western blot.
E8. The method of any one of E1-E7, wherein the rAAV vector has an in vivo potency of at least 5%, at least 10%, at least 15%, at least 20%, at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard.
E9. The method of E8, wherein the in vivo potency is measured in an animal model.
E10. The method of any one of E1-E9, wherein the insect cell is cultured for 24 hours, 2 days, 3 days, 4 days, 4.1 days, 4.2 days, 4.3 days, 4.4 days, 4.5 days, 4.6 days, 4.7 days, 4.8 days, 4.9 days, 5 days, 5.1 days, 5.2 days, 5.3 days, 5.4 days, 5.5 days, 6 days, 7 days, 8 days, 9 days or 10 days prior to recovering the rAAV vector from the insect cell.

E11. The method of any one of E1-E10, wherein the insect cell is cultured for at least 4.1 days but no more than 10 days prior to recovering the rAAV vector from the insect cell.
E12. The method of any one of E1-E10, wherein the insect cell is cultured for about 4 days, 4.1 days, 4.2 days, 4.3 days, 4.4 days, 4.5 days, 4.6 days, 4.7 days, 4.8 days, 4.9 days, 5 days, 5.1 days, 5.2 days, 5.3 days, 5.4 days, or 5.5 days prior to recovering the rAAV vector from the insect cell.
E13. The method of any one of E1-E12, wherein the insect cell is cultured for about 96 hours to about 128 hours prior to recovering the rAAV vector from the insect cell, or about 108±5 hours prior to recovering the rAAV vector from the insect cell.
E14. The method of any one of E1-E13, wherein the genome titer measured prior to recovery of rAAV vector from the insect cell is at least 1×10¹⁰ viral genomes (vg)/ml.
E15. The method of any one of E1-E14, wherein the genome titer measured after recovery of rAAV vector from the insect cell is at least 5×10⁹ viral genomes (vg)/ml.
E16. The method of E14 or E15, wherein the genome titer is measured by quantitative polymerase chain reaction (qPCR).
E17. The method of any one of E1-E16, wherein the insect cell is contacted with:
(i) one or two helper recombinant baculovirus(es), each baculovirus comprising a heterologous sequence encoding AAV Rep proteins and/or AAV Cap proteins, and
(iii) a vector recombinant baculovirus comprising a heterologous sequence encoding a transgene between two AAV inverted terminal repeats (ITRs).
E18. The method of E17, wherein suitable conditions for culturing the insect cell that produce rAAV vector with no more than 15% clipping between amino acid residues 115G and 116R on VP1 protein or rAAV vector with no more than 65% clipping between amino acid residues 189G and 190E and no more than 15% clipping between amino acid residues 115G and 116R on VP1 and VP2 proteins of wild-type AAV6 or the corresponding amino acids in the VP1 and VP2 proteins of another AAV serotype comprise:
   (i) temperature at which the insect cell is cultured,
   (ii) amount of helper recombinant baculovirus contacted with the insect cell,
   (iii) amount of vector recombinant baculovirus vector contacted with the insect cell, and/or
   (iv) amount of dissolved oxygen in the cell culture medium.
E19. The method of any one of E1-E18, wherein the insect cell is cultured at a temperature lower than 37° C.
E20. The method of E19, wherein the insect cell is cultured at a temperature of about 25° C., about 26° C., about 27° C., about 28° C., about 29° C., about 30° C. or about 31° C.
E21. The method of E20, wherein the insect cell is cultured at a temperature of about 28° C.
E22. The method of any one of E18-E21, wherein the amount of helper recombinant baculovirus contacted with the insect cell is between about 0.0022% to about 0.0178% volume relative to the total culture volume.
E23. The method of any one of E18-E22, wherein the amount of vector recombinant baculovirus contacted with the insect cell is between about 0.0022% to about 0.0178% volume relative to the total culture volume.
E24. The method of any one E18-E23, wherein the amount of dissolved oxygen in the culture medium is about 20% to about 100% of air saturation.
E25. The method of any one of E1-E24, wherein the insect cell is cultured in a cell culture medium, and wherein the volume of the cell culture medium is at least 2 L, at least 10 L, at least 250 L, or at least 2000 L.
E26. The method of any one of E1-E25, wherein the clipping on VP1 and VP2 proteins is measured by capillary gel electrophoresis (CGE), mass spectrometry (including multi-attribute mass spectrometry), and/or Western blot assays.
E27. The method of any one of E1-26, wherein the insect cell is Sf9 cell, Sf21 cell or Hi5 cell.
E28. The method of any one of E1-E27, wherein the insect cells are in suspension culture.
E29. The method of any one of E1-E27, wherein the insect cells are adherent.
E30. The method of any one of E1-E29, wherein the insect cells are grown or maintained in a serum-free culture medium.
E31. The method of any one of E1-E30, wherein the insect cells are grown or maintained in roller bottles or expanded roller bottles.
E32. The method of any one of E1-E30, wherein the insect cells are grown in bioreactors.
E33. The method of any one of E1-E30, wherein the insect cells are grown in bags or flasks.
E34. The method of claim 32, wherein the insect cells are grown in a WAVE bioreactor.
E35. The method of E32, wherein the cells are grown in a stirred tank bioreactor.
E36. The method of any one of E17-E35, wherein the transgene encodes a wild type or functional variant blood clotting factor, mini-dystrophin, C1 esterase inhibitor, copper transporting P-type ATPase (ATP7B), copper-zinc superoxide dismutase 1 (SOD1) or myosin binding protein C3.
E37. The method of E36, wherein the wild type of functional variant blood clotting factor is Factor VII, Factor VIII or Factor IX.
E38. The method of E37, wherein the wild type or functional variant blood clotting factor is Factor VIII.
E39. The method of any one of E1-E38, wherein the rAAV is rAAV1, rAAV3a, rAAV3b, rAAV6, or rAAV8.
E40. A composition comprising purified, recombinant adeno-associated virus (rAAV) vector with no more than 15% clipping between amino acid residues 115G and 116R on VP1 protein of wild-type AAV6 or the corresponding amino acids in the VP1 protein of another AAV serotype.
E41. A composition comprising purified, recombinant adeno-associated virus (rAAV) vector with no more than 15% clipping between amino acid residues 115G and 116R and no more than 65% clipping between amino acid residues 189G and 190E on VP1 and VP2 proteins of wild-type AAV6, or the corresponding amino acids in the VP1 and VP2 proteins of another AAV serotype.
E42. The composition of any one of E40-E41, wherein the rAAV is rAAV1, rAAV3a, rAAV3b, rAAV6, or rAAV8.
E43. The composition of any one of E40-E42, wherein the rAAV vector comprises a transgene of interest.
E44. The composition of E43, wherein the transgene of interest encodes a wild type or functional variant blood clotting factor, mini-dystrophin, C1 esterase inhibitor, copper transporting P-type ATPase (ATP7B), copper-zinc superoxide dismutase 1 (SOD1) or myosin binding protein C3.
E45. The composition of E44, wherein the wild type of functional variant blood clotting factor is Factor VII, Factor VIII or Factor IX.
E46. The composition of E45, wherein the wild type or functional variant blood clotting factor is Factor VIII.
E47. The composition of any one of E40-E46, wherein the rAAV vector has an in vitro potency of at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard.
E48. The composition of E47, wherein the in vitro potency is measured using colorimetric assay, a chromogenic assay, an ELISA-based assay, quantitative PCR, and/or Western blot.
E49. The composition of any one of E40-E48 wherein the rAAV vector has an in vivo potency of at least 5%, at least 10%, at least 15%, at least 20%, at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard.
E50. The composition of E49, wherein the in vivo potency is measured in an animal model.
E51. The composition of any one of E40-E50, wherein the clipping on VP1 and VP2 proteins is measured by capillary gel electrophoresis (CGE), mass spectrometry (including multi-attribute mass spectrometry), and/or Western blot assays.
E52. A composition comprising purified, recombinant adeno-associated virus 6 (rAAV6) vector comprising a transgene encoding a wild type or functional variant blood clotting factor VIII having an in vitro potency of about 10%-500% as compared to a reference standard with no more than 15% clipping between amino acid residues 115G and 116R on VP1 protein.
E53. A composition comprising purified, recombinant adeno-associated virus 6 (rAAV6) vector comprising a transgene encoding a wild type or functional variant blood clotting factor VIII having an in vitro potency of about 10%-500% as compared to a reference standard with no more than 15% clipping between amino acid residues 115G and 116R and no more than 65% clipping between amino acid residues 189G and 190E on VP1 and VP2 proteins.
E54. A method for producing recombinant adeno-associated virus 6 (rAAV6) vector comprising blood clotting Factor VIII, the method comprising:
   (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and
   (ii) culturing the Sf9 cell under suitable conditions for 108±5 hours to produce rAAV6 vector with no more than 15% clipping between VP1 amino acid residues that correspond to Gly115 and Arg116 of wild-type AAV6.
E55. A method for producing recombinant adeno-associated virus 6 (rAAV6) vector comprising blood clotting Factor VIII, the method comprising:
   (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and
   (ii) culturing the Sf9 cell under suitable conditions for 108±5 hours to produce rAAV6 vector with no more than 65% clipping between VP1 and VP2 amino acid residues that correspond to Gly189 and Glu190 and no more than 15% clipping between VP1 amino acid residues that correspond to Gly115 and Arg116 of wild-type AAV6.
E56. A method for producing recombinant adeno-associated virus 6 (rAAV6) vector comprising blood clotting Factor VIII, the method comprising:
   (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and
   (ii) culturing the Sf9 cell under suitable conditions for 108±5 hours to produce rAAV6 vector having an in vitro potency that is between 50-150% as compared to a reference standard with no more than 15% clipping between VP1 amino acid residues that correspond to Gly115 and Arg116 of wild-type AAV6.
E57. A method for producing recombinant adeno-associated virus 6 (rAAV6) vector comprising blood clotting Factor VIII, the method comprising:
   (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and
   (ii) culturing the Sf9 cell under suitable conditions for 108±5 hours to produce rAAV6 vector having an in vitro potency that is between 50-150% as compared to a reference standard with no more than 65% clipping between VP1 and VP2 amino acid residues that correspond to Gly189 and Glu190 and no more than 15% clipping between VP1 amino acid residues that correspond to Gly115 and Arg116 of wild-type AAV6.

### BRIEF DESCRIPTION OF THE DRAWINGS

FIG. 1 shows a graph demonstrating the negative correlation between in vitro potency and batch duration post infection at the 2000 L scale.

FIG. 2 shows a graph demonstrating the inverse relationship between in vitro potency and clipping of VP1/VP2 capsid proteins between amino acid residues G189 and E190.

FIG. 3 shows a graph demonstrating that the VP1/VP2 clipping increases as a function of batch duration post infection.

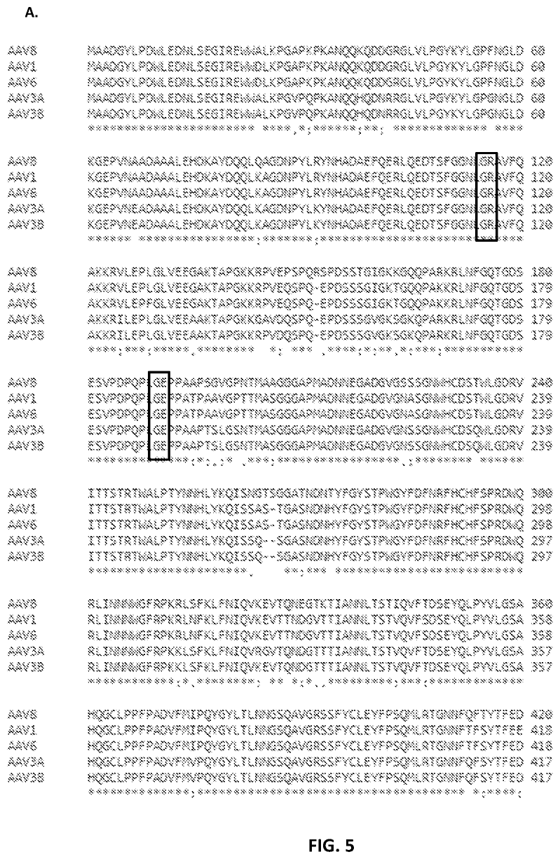

FIG. 4 is a schematic representation of the assay used to measure FVIII activity.

FIG. 5 is an alignment of wild type AAV1 (SEQ ID NO: 1), AAV3A (SEQ ID NO: 2), AAV3B (SEQ ID NO: 3), AAV6 (SEQ ID NO: 4) and AAV8 (SEQ ID NO: 5).

FIG. 6 depicts graphs showing the quantitative relationship between in vitro Factor VIII activity and (A) stirred tank reactor (STR) temperature, (B) amount of dissolved oxygen expressed as percentage of air saturation, (C) helper recombinant baculovirus volume, (D) vector recombinant baculovirus volume, and (E) batch duration post-infection.

### DETAILED DESCRIPTION

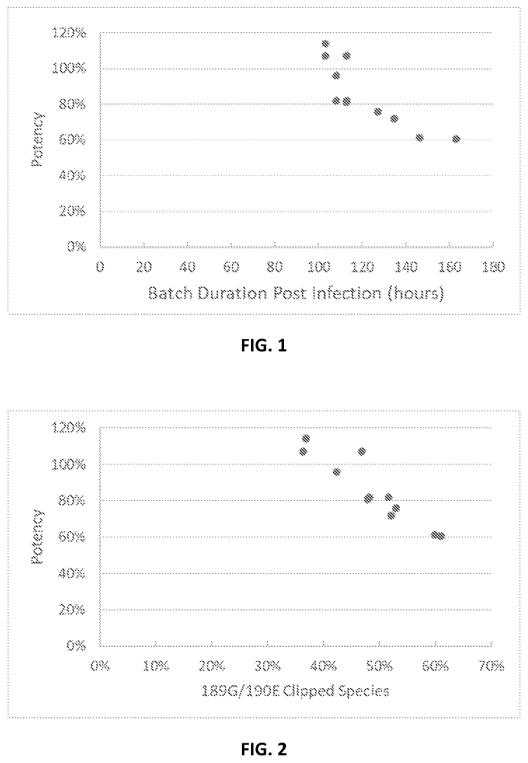

Analysis of rAAV vector produced in insect cells using the baculovirus expression vector (BEV) system demonstrated an inverse correlation between the in vitro potency values of the rAAV vector and time post-infection (i.e., number of days after contacting the insect cells with the recombinant baculoviruses prior to recovery of the rAAV vector, also called batch duration post-infection herein). Specifically, it was observed that the in vitro potency of rAAV vector decreased with time post-infection, notably between about 103 hours and 163 hours (see, for example, FIG. 1).

Further studies conducted to understand the cause of this change in in vitro potency led to the identification of an attribute—clipped forms of AAV capsid proteins, i.e., VP1 and VP2 proteins. The data demonstrated an inverse correlation between the clipped VP1 and VP2 protein levels and the relative in vitro potency, where in vitro potency decreased as the level of clipped VP1 and VP2 proteins increased with time post-infection. This increase in clipped VP1 and VP2 proteins with time post-infection and the concomitant decrease in in vitro potency was observed both at large-scale production (2000 L) and small-scale production (2 L, 10 L or 200 L).

Accordingly, the present disclosure provides methods for producing rAAV vector by optimizing the time post-infection so that the rAAV vector produced has a minimal level of clipped VP1 and VP2 proteins and maximal in vitro potency. The disclosure also provides compositions comprising purified rAAV vector with minimal clipped VP1 and VP2 proteins and maximal in vitro potency.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The present disclosure may be understood more readily by reference to the following detailed description of exemplary embodiments of the invention and the Examples included therein.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

### Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

As used herein, the term “about,” or “approximately” refers to a measurable value such as (but not limited to) an amount of clipped VP1 and/or VP2 proteins, in vitro potency, time post-infection, temperature, dose, genome titer, amount of helper recombinant baculovirus, amount of vector recombinant baculovirus, the biological activity, length of a polynucleotide or polypeptide sequence and the like, and is meant to encompass variations of 25%, 20%, 19%, 18%, 17%, 16%, 15%, 14%, 13%, 12%, 11%, 10%, 9%, 8%, 7%, 6%, 5%, 4%, 3%, 2% 1%, 0.5% or even 0.1%, in either direction (greater than or less than) of the specified amount unless otherwise stated, otherwise evident from the context, or except where such number would exceed 100% of a possible value.

As used herein, the term “and/or” refers to and encompasses any and all possible combinations of one or more of the associated listed items, as well as the lack of combinations when interpreted in the alternative (“or”).

As used herein, the terms “adeno-associated virus” and/or “AAV” refer to a parvovirus with a linear single-stranded DNA genome and variants thereof. The term covers all subtypes and both naturally occurring and recombinant forms, except where required otherwise. The wild-type genome comprises 4681 bases (Berns and Bohenzky (1987) Advances in Virus Research 32:243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap” or “AAV structural proteins”) genes, respectively. AAV rep and cap may also be referred to herein as AAV “packaging genes.” These genes code for the viral proteins involved in replication and packaging of the viral genome.

In wild type AAV virus, three capsid genes VP1, VP2 and VP3 overlap each other within a single open reading frame and alternative splicing leads to production of VP1, VP2 and VP3. (Grieger and Samulski (2005) J. Virol. 79(15):9933-9944.) A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of SEQ ID NO:123, VP2 comprises amino acid residues 138 to 736 of SEQ ID NO:123, and VP3 comprises amino acid residues 203 to 736 of SEQ ID NO:123. As uses herein, the terms “AAV capsid” or “AAV Cap” or “cap” refers to AAV capsid proteins VP1, VP2 and/or VP3, and variants and analogs thereof.

At least four viral proteins are synthesized from the AAV rep gene, Rep 78, Rep 68, Rep 52 and Rep 40, and are named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a rep may comprise nucleotides from an AAV2 serotype and nucleotides from an AA3 serotype (Rabinowitz et al. (2002) J. Virology 76(2):791-801).

As used herein the terms “recombinant adeno-associated virus vector,” “rAAV” and/or “rAAV vector” refer to an AAV comprising a vector genome wherein the rep and/or cap genes of the wild type AAV virus genome have been removed from the virus genome and replaced with a polynucleotide sequence not of, or not entirely of, AAV origin (e.g., a polynucleotide heterologous to AAV). Where the rep and/or cap genes of the canonical AAV have been removed or are not present (and where the flanking ITRs are typically derived from ITRs from a different serotype, such as, but not limited to AAV2 ITRs where the capsid is not AAV2), the nucleic acid within the AAV, including any ITR and any nucleic acid between them, is referred to as the “vector genome.” Therefore, the term rAAV vector encompasses an rAAV viral particle that comprises a capsid and a heterologous nucleic acid, i.e., a nucleic acid not originally present in the capsid in nature, and herein also referred to as a “vector genome.” Thus, a “rAAV vector genome” (or “vector genome”) refers to a heterologous polynucleotide sequence (including at least one ITR, typically, but not necessarily, an ITR not associated with the original nucleic acid present in the original AAV) that may, but need not, be contained within an AAV capsid. An rAAV vector genome may be double-stranded (dsAAV), single-stranded (ssAAV) and/or self-complementary (scAAV).

As used herein, the terms “rAAV vector,” “rAAV viral particle” and/or “rAAV vector particle” refer to an AAV capsid comprised of at least one AAV capsid protein (though typically all of the capsid proteins, e.g., VP1, VP2 and VP3, or variant thereof, of an AAV are present) and containing a vector genome comprising a heterologous nucleic acid sequence not originally present in the original AAV capsid. These terms are to be distinguished from an “AAV viral particle” or “AAV virus” that is not recombinant wherein the capsid contains a virus genome encoding rep and cap genes and which AAV virus is capable of replicating if present in a cell also comprising a helper virus, such as an adenovirus and/or herpes simplex virus, and/or required helper genes therefrom. Thus, production of an rAAV vector particle necessarily includes production of a recombinant vector genome using recombinant DNA technologies, as such, which vector genome is contained within a capsid to form an rAAV vector, rAAV viral particle, or an rAAV vector particle.

The genomic sequences of various serotypes of AAV, as well as the sequences of the inverted terminal repeats (ITRs), rep proteins, and capsid subunits, both existing in nature and/or mutants and variants thereof, are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC_001863 (AAV-3B), NC-001829 (AAV-4), U89790 (AAV-4), NC-006152 (AAV-5), AF028704 (AAV-6), AF513851 (AAV-7), AF513852 (AAV-8), and NC-006261 (AAV-8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379, WO 2014/194132, WO 2015/121501; and U.S. Pat. Nos. 6,156,303 and 7,906,111.

As used herein, the term “ameliorate” means a detectable or measurable improvement in a subject's disease, disorder or condition, or symptom thereof, or an underlying cellular response. A detectable or measurable improvement includes a subjective or objective decrease, reduction, inhibition, suppression, limit or control in the occurrence, frequency, severity, progression or duration of, complication cause by or associated with, improvement in a symptom of, or a reversal of a disease, disorder or condition.

As used herein, the term “coding sequence” or “encoding nucleic acid” refers to a nucleic acid sequence which encodes a protein or polypeptide and denotes a sequence which is transcribed (in the case of DNA) and translated (in the case of mRNA) into a polypeptide in vitro or in vivo when placed under the control of (operably linked to) appropriate regulatory sequences. Boundaries of a coding sequence are generally determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

As used herein, the term “chimeric” refers to a viral capsid, with capsid sequences from different parvoviruses, preferably different AAV serotypes, as described in Rabinowitz et al., U.S. Pat. No. 6,491,907, the disclosure of which is incorporated in its entirety herein by reference. See also Rabinowitz et al. (2004) J. Virol. 78(9):4421-4432. In some embodiments, a chimeric viral capsid is an AAV2.5 capsid described in WO 2006/066066, AAV2i8 described in WO 2010/093784, AAV2G9 and AAV8G9 described in WO 2014/144229, and AAV9.45 (Pulicherla et al. (2011) Molecular Therapy 19(6):1070-1078), AAV-NP4, NP22, NP66, AAV-LK01 through AAV-LK019 described in WO 2103/029030, RHM4-1 and RHM15-1 through RHM5-6 described in WO 205/013313, AAV-DJ, AAV-DJ/8, AAV-DJ/9 described in WO 2007/120542.

As used herein, the term “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or are of a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid, glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine. A “conservative substitution” also includes the use of a substituted amino acid in place of an unsubstituted parent amino acid.

As used herein, the term “flanked,” refers to a sequence that is flanked by other elements and indicates the presence of one or more flanking elements upstream and/or downstream, i.e., 5′ and/or 3′, relative to the sequence. The term “flanked” is not intended to indicate that the sequences are necessarily contiguous. For example, there may be intervening sequences between a nucleic acid encoding a transgene and a flanking element. A sequence (e.g., a transgene) that is “flanked” by two other elements (e.g., ITRs), indicates that one element is located 5′ to the sequence and the other is located 3′ to the sequence; however, there may be intervening sequences there between.

As used herein, the term “fragment” refers to a material or entity that has a structure that includes a discrete portion of the whole but lacks one or more moieties found in the whole. In some embodiments, a fragment consists of a discrete portion. In some embodiments, a fragment consists of or comprises a characteristic structural element or moiety found in the whole. In some embodiments, a polymer fragment comprises, or consists of, at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., amino acid residues, nucleotides) found in the whole polymer.

As used herein, the term “functional” refers to a biological molecule in a form in which it exhibits a property and/or activity by which it is characterized. A biological molecule may have two functions (i.e., bifunctional) or many functions (i.e., multifunctional).

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the term “heterologous” or “exogenous” nucleic acid refers to a nucleic acid inserted into a vector (e.g., rAAV vector or a recombinant baculovirus) for purposes of vector mediated transfer/delivery of the nucleic acid into a cell. Heterologous nucleic acids are typically distinct from the vector (e.g., AAV, baculovirus) nucleic acid, that is, the heterologous nucleic acid is non-native with respect to the viral (e.g., AAV, baculovirus) nucleic acid found in in nature. Once transferred (e.g., transduced) or delivered into a cell, a heterologous nucleic acid, contained within a vector, can be expressed (e.g., transcribed and translated if appropriate). Alternatively, a transferred (transduced) or delivered heterologous nucleic acid in a cell, contained within the vector, need not be expressed. Although the term “heterologous” is not always used herein in reference to a nucleic acid, reference to a nucleic acid even in the absence of the modifier “heterologous” is intended to include a heterologous nucleic acid. For example, a heterologous nucleic acid would be a nucleic acid encoding a wild type or functional variant blood clotting factor, mini-dystrophin, C1 esterase inhibitor, copper transporting P-type ATPase (ATP7B), copper-zinc superoxide dismutase 1 (SOD1) or myosin binding protein C3.

As used herein, the term “homologous,” or “homology,” refers to two or more reference entities (e.g., a nucleic acid or polypeptide sequence) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 95% to 99% of the sequence. The extent of homology (identity) between two sequences can be ascertained using computer program or mathematical algorithm. Such algorithms that calculate percent sequence homology (or identity) generally account for sequence gaps and mismatches over the comparison region or area. Exemplary programs and algorithms are provided below.

As used herein, the terms “host cell,” “host cell line,” and “host cell culture” are used interchangeably and refers to a cell into which an exogenous nucleic acid has been introduced, and includes the progeny of such a cell. A host cell includes a “transfectant,” “transformant,” “transformed cell,” and “transduced cell,” which includes the primary transfected, transformed or transduced cell, and progeny derived therefrom, without regard to the number of passages. In some embodiments, a host cell is a packaging cell for production of an rAAV vector such as Sf9 or Sf21 cell.

As used herein, the term “identity” or “identical to” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 99% or more identical.

Calculation of the percent identity of two nucleic acid or polypeptide sequences, for example, can be performed by aligning two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. Nucleotides at corresponding positions are then compared. When a position in a first sequence is occupied by the same residue (e.g., nucleotide or amino acid) as the corresponding position in a second sequence, then the molecules are identical at that position. The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, or homology, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Also, of interest is the BestFit program using the local homology algorithm of Smith and Waterman (1981, Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

As used herein, the terms “increase,” improve” or “decrease”, “reduce” indicate values that are relative to a baseline measurement. For example, an increase or improvement in in vitro potency may be measured relative to the in vitro potency value of rAAV vector not produced by the methods described herein.

As used herein, the terms “inverted terminal repeat,” “ITR,” “terminal repeat,” and “TR” refer to palindromic terminal repeat sequences at or near the ends of the AAV genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce an rAAV vector (also referred to herein as “rAAV vector particle” or “rAAV viral particle”) comprising the vector genome.

As used herein, the term “isolated” refers to a substance or composition that is 1) designed, produced, prepared, and or manufactured by the hand of man and/or 2) separated from at least one of the components with which it was associated when initially produced (whether in nature and/or in an experimental setting). Generally, isolated compositions are substantially free of one or more materials with which they normally associate with in nature, for example, one or more protein, nucleic acid, lipid, carbohydrate and/or cell membrane. The term “isolated” does not exclude man-made combinations, for example, a recombinant nucleic acid, a recombinant vector genome (e.g., rAAV vector genome), an rAAV vector particle (e.g., such as, but not limited to, an rAAV vector particle comprising an AAV6 capsid) that packages, e.g., encapsidates, a vector genome and a pharmaceutical formulation. The term “isolated” also does not exclude alternative physical forms of the composition, such as hybrids/chimeras, multimers/oligomers, modifications (e.g., phosphorylation, glycosylation, lipidation), variants or derivatized forms, or forms expressed in host cells that are man-made.

Isolated substances or compositions may be separated from about 10%, about 20%, about 30%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without including such carriers or excipients.

As used herein, the terms “nucleic acid sequence,” “nucleotide sequence,” and “polynucleotide” refer interchangeably to any molecule composed of or comprising monomeric nucleotides connected by phosphodiester linkages. A nucleic acid may be an oligonucleotide or a polynucleotide. Nucleic acid sequences are presented herein in the direction from the 5′ to the 3′ direction. A nucleic acid sequence (i.e., a polynucleotide) of the present disclosure can be a deoxyribonucleic acid (DNA) molecule or ribonucleic acid (RNA) molecule and refers to all forms of a nucleic acid such as, double stranded molecules, single stranded molecules, small or short hairpin RNA (shRNA), micro RNA, small or short interfering RNA (siRNA), trans-splicing RNA, antisense RNA, messenger RNA, transfer RNA, ribosomal RNA. Where a polynucleotide is a DNA molecule, that molecule can be a gene, a cDNA, an antisense molecule or a fragment of any of the foregoing molecules. Nucleotides are indicated herein by a single letter code: adenine (A), guanine (G), thymine (T), cytosine (C), inosine (I) and uracil (U). A nucleotide sequence may be chemically modified or artificial. Nucleotide sequences include peptide nucleic acids (PNA), morpholinos and locked nucleic acids (LNA), as well as glycol nucleic acids (GNA) and threose nucleic acids (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′-P5′-phosphoramidates, and oligoribonucleotide phosphorothioates and their 2′-0-allyl analogs and 2′-0-methylribonucleotide methylphosphonates which may be used in a nucleotide sequence of the disclosure.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, an rAAV vector genome, an expression vector, etc.), that is, a nucleic acid molecule designed to deliver exogenously created DNA into a host cell.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact.

As used herein, the terms “polypeptide,” “protein,” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be but are not required to be identical to the endogenous protein that is defective, or whose expression is insufficient, or deficient in a subject treated with gene therapy.

As used herein, the term “prevent” or “prevention” refers to delay of onset, and/or reduction in frequency and/or severity of one or more sign or symptom of a particular disease, disorder or condition (e.g., Canavan disease). In some embodiments, prevention is assessed on a population basis such that an agent is considered to “prevent” a particular disease, disorder or condition if a statistically significant decrease in the development, frequency and/or intensity of one or more sign or symptom of the disease, disorder or condition is observed in a population susceptible to the disease, disorder or condition. Prevention may be considered complete when onset of disease, disorder or condition has been delayed for a predefined period of time.

As used herein, the term “recombinant,” refers to a vector, polynucleotide (e.g., a recombinant nucleic acid), polypeptide or cell that is the product of various combinations of cloning, restriction or ligation steps (e.g. relating to a polynucleotide or polypeptide comprised therein), and/or other procedure that results in a construct that is distinct from a product found in nature. A recombinant virus or vector (e.g., rAAV vector, recombinant baculovirus) comprises a vector genome comprising a recombinant nucleic acid (e.g., a nucleic acid comprising a transgene and one or more regulatory elements). The terms respectively include replicates of the original polynucleotide construct and progeny of the original virus construct.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from or is susceptible to a disease, disorder or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. For example, but not limited to, the subject may be suffering from hemophilia (e.g., hemophilia A or B), Duchenne's Muscular Dystrophy (DMD), Wilson's disease, Amyotrophic Lateral Sclerosis (ALS), Hereditary Angioedema (HAE), Pompe Disease, or hypertrophic cardiomyopathy caused by MYBPC3 mutations.

As used herein, the term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

As used herein, the term “symptoms are reduced” or “reduce symptoms” refers to when one or more symptoms of a particular disease, disorder or condition is reduced in magnitude (e.g., intensity, severity etc.) and/or frequency. For purposes of clarity, a delay in the onset of a particular symptom is considered one form of reducing the frequency of that symptom.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is a wild type or functional variant blood clotting factor, mini-dystrophin, C1 esterase inhibitor, copper transporting P-type ATPase (ATP7B), copper-zinc superoxide dismutase 1 (SOD1) or myosin binding protein C3.

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector, recombinant baculovirus). A transgene may be operably linked to a control sequence, such as a promoter. It will be appreciated by those of skill in the art that expression control sequences can be selected based on ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example a blood clotting factor such Factor VIII or Factor IX, and an exemplary promoter is one not operable linked to a nucleotide encoding Factors VIII or IX in nature. Such a non-endogenous promoter can include a minimal transthyretin promoter, among many others known in the art.

A nucleic acid of interest can be introduced into a host cell by a wide variety of techniques that are well-known in the art, including transfection and transduction.

“Transfection” is generally known as a technique for introducing an exogenous nucleic acid into a cell without the use of a viral vector. As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell (e.g., a CHO cell, Pro10 cell, HEK293 cell, Sf9 cell) comprising an expression plasmid/vector for producing a recombinant AAV vector. In some embodiments, a transfected cell (e.g., a packing cell) may comprise a plasmid comprising a transgene, a plasmid comprising an AAV rep gene and an AAV cap gene and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector to a cell (e.g., a rAAV vector transfer to a target cell, or a recombinant baculovirus transfer of its heterologous nucleotide to an insect cell). A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide.

Cells that may be transduced include a cell of any tissue or organ type, or any origin (e.g., mesoderm, ectoderm or endoderm). Non-limiting examples of cells include liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells, exocrine cells), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblast, myocytes), synoviocytes, chondrocytes, osteoclasts, epithelial cells, endothelial cells, salivary gland cells, inner ear nervous cells or hematopoietic (e.g., blood or lymph) cells.

As used herein, the terms “treat,” “treating” or treatment refer to administration of a therapy that partially or completely alleviates, ameliorates, relieves, inhibits, delays onset of, reduces severity of, and/or reduces incidence of one or more symptoms, features, and/or causes of a particular disease, disorder, and/or condition.

As used herein, the term “vector” refers to a plasmid, virus (e.g., an rAAV, recombinant baculovirus), cosmid, bacmid or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, a vector nucleic acid comprises an AAV Cap expression cassette and/or an AAV Rep expression cassette. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene.

Plasmid vectors may be referred to herein as “expression vectors.” In some embodiments, the vector is an insect-cell compatible vector (i.e., the vector facilitates the transduction of insect cells with a heterologous nucleic acid). In some embodiments, the insect-cell compatible vector is a recombinant baculovirus (such as, for example, a bacmid shuttle vector (Luckow, V, et al., J. Virol. 67, 4566-4579, 1993) The recombinant baculovirus is preferentially capable of replication in an insect cell such as StN cells and in a prokaryotic cell such as E. coli. Any viral baculovirus that contains a BAC replicon may be used. In a particular embodiment, the recombinant baculovirus genome is a bacmid. Suitable baculoviruses that can be used in the methods described herein include the Autographa californica (multicapsid nucleopolyhedrovirus (AcMNPV), Bombyxmori (Bm)NPV, Helicoverpa armigera (Hear) NPV) or Spodoptera exigua (Se) MNPV. In particular, the recombinant baculovirus may be derived from the AcMNPV clone C6 (genomic sequence: Genbank accession no NC_001623.1) or E2 (Smith & amp; Summers, 1979).

As used herein, the term “vector genome” refers to a recombinant nucleic acid sequence that is packaged or encapsidated to form an rAAV vector or a recombinant baculovirus. Typically, a vector genome includes a heterologous polynucleotide sequence, e.g., a transgene, regulatory elements, ITRs not originally present in the AAV or baculovirus In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is typically referred to as the “plasmid backbone,” which is important for cloning, selection and amplification of the plasmid, a process that is needed for propagation of recombinant viral vector production, but which is not itself packaged or encapsidated into an rAAV vector.

As used herein, the term “viral vector” generally refers to a viral particle that functions as a nucleic acid delivery vehicle and which comprises a vector genome (e.g., comprising a transgene instead of a nucleic acid encoding an AAV rep and cap) packaged within the viral particle (i.e., capsid) and includes, for example, lenti- and parvo-viruses, including AAV serotypes and variants (e.g., rAAV vectors).

AAV and rAAV Vectors

### AAV

The present disclosure relates to compositions and methods for producing rAAV vectors in insect cells. As discussed supra, the terms “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV type 2i8 (AAV2i8), NP4, NP22, NP66, DJ, DJ/8, DJ/9, LK3, RHM4-1, among many others. “Primate AAV” refers to AAV that infect primates, “non-primate AAV” refers to AAV that infect non-primate mammals, “bovine AAV” refers to AAV that infect bovine mammals, and so on. Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, e.g., AAV, that are not serologically distinct but that may be within a subgroup or a variant of a given serotype.

A comprehensive list and alignment of amino acid sequences of capsids of known AAV serotypes is provided by Marsic et al. (2014) Molecular Therapy 22(11):1900-1909, especially at supplementary FIG. 1.

Genomic sequences of various serotypes of AAV, as well as sequences of the native terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC 001729 (AAV3), NC_001863 (AAV3B), NC 001829 (AAV4), U89790 (AAV4), NC_006152 (AAV5), NC_001862 (AAV6), AF513851 (AAV7), AF513852 (AAV8), and NC_006261 (AAV8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111. For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in a 1:1:10 ratio in the capsid. That is, for AAVs, VP1 is the full-length protein and VP2 and VP3 are progressively shorter versions of VP1, with increasing truncation of the N-terminus relative to VP1.

### Recombinant AAV

As discussed supra, a “recombinant adeno-associated virus” or “rAAV” is distinguished from a wild-type AAV by replacement of all or part of the endogenous viral genome with a non-native sequence. Incorporation of a non-native sequence within the virus defines the viral vector as a “recombinant” vector, and hence a “rAAV vector.” An rAAV vector can include a heterologous polynucleotide (i.e., transgene”) encoding a desired protein or polypeptide (e.g., a blood clotting factor). A recombinant vector sequence may be encapsidated or packaged into an AAV capsid and referred to as an “rAAV vector,” an “rAAV vector particle,” “rAAV viral particle” or simply a “rAAV.”

For the production of an rAAV vector, the desired ratio of VP1:VP2:VP3 is in the range of about 1:1:1 to about 1:1:100, preferably in the range of about 1:1:2 to about 1:1:50, more preferably in the range of about 1:1:5 to about 1:1:20. Although the desired ratio of VP1:VP2 is 1:1, the ratio range of VP1:VP2 could vary from 1:50 to 50:1.

The transgene included in the rAAV vector may be flanked by at least one, and sometimes by two, AAV terminal repeat sequences (e.g., inverted terminal repeats (ITRs)). The transgene flanked by ITRs (also referred to herein as a “vector genome”) typically encodes a polypeptide of interest, or a gene of interest (“GOI”), such as a target for therapeutic treatment (e.g., a nucleic acid encoding a blood clotting factor, such Factor VIII or Factor IX for the treatment of hemophilia A or B). Delivery or administration of an rAAV vector to a subject (e.g. a patient) provides encoded proteins and peptides to the subject. Thus, an rAAV vector can be used to transfer/deliver a transgene for expression for, e.g., treating a variety of diseases, disorders and conditions.

Proteins encoded by the transgene include therapeutic proteins. Non-limiting examples include a blood clotting factor (e.g., Factor XIII, Factor IX, Factor X, Factor VIII, Factor VIIa, or protein C), mini-dystrophin, C1 esterase inhibitor, copper transporting P-type ATPase (ATP7B), copper-zinc superoxide dismutase 1 (SOD1), myosin binding protein C3 (MYBPC3), apoE2, arginino succinate synthase, acid alpha-glucosidase, β-Glucocerebrosidase, a-galactosidase, CI inhibitor serine protease inhibitor, CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, a-globin, spectrin, a-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor-3 and -4, brain-derived neurotrophic factor, glial derived growth factor, transforming growth factor a and β, etc.), a cytokine (e.g., a-interferon, β-interferon, interferon-γ, interleukin-2, interleukin-4, interleukin 12, granulocyte-macrophage colony stimulating factor, lymphotoxin, etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g., that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDRI, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB 1 (Connexin 26 deafness), ACHM 2, 3 and 4 (Achromatopsia), PKD-1 or PKD-2 (Polycystic kidney disease), gene deficiencies causative of lysosomal storage diseases (e.g., sulfatases, N-acetylglucosamine-1-phosphate transferase, cathepsin A, GM2-AP, NPC1, VPC2, Sphingolipid activator proteins, etc.), one or more zinc finger nucleases for genome editing, or donor sequences used as repair templates for genome editing. In some embodiments, the transgene encodes Factor VIII. In some embodiments, the transgene comprises SEQ ID NO: 9.

In some embodiments, the transgene contained within the rAAV vector produces a transcript when transcribed. Such transcripts can be RNA, such as inhibitory RNA (RNAi, e.g., small or short hairpin (sh)RNA, microRNA (miRNA), small or short interfering (si)RNA, trans-splicing RNA, or antisense RNA).

Non-limiting examples include inhibitory nucleic acids that inhibit expression of: huntington (HTT) gene, a gene associated with dentatorubropallidolusyan atropy (e.g., atrophin 1, ATN1); androgen receptor on the X chromosome in spinobulbar muscular atrophy, human Ataxin-1, -2, -3, and -7, Cav2.1 P/Q voltage-dependent calcium channel is encoded by the (CACNA1A), TATA-binding protein, Ataxin 8 opposite strand, also known as ATXN80S, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7,also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Reddl also known as DAN damage-inducible transcript 4 protein, in diabetic macular oedma (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

rAAV vector genomes generally retain 145 base ITRs in cis to the heterologous nucleic acid sequence that replaced the viral rep and cap genes. Such ITRs are necessary to produce a recombinant AAV vector; however, modified AAV ITRs and non-AAV terminal repeats including partially, or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. An rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV. In some embodiments, the ITRs are derived from AAV2.

An rAAV vector of the disclosure may comprise an ITR from an AAV serotype (e.g., wild-type AAV2, a fragment or variant thereof) that differs from the serotype of the capsid (e.g., AAV1, AAV6, AAV8, or Olig001). Such an rAAV vector comprising at least one ITR from one serotype, but comprising a capsid from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, an rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from an rAAV vector can be hindered by the need to convert a single stranded rAAV genome (ssAAV) into double-stranded DNA prior to expression. This step is circumvented by using a self-complementary AAV genome (scAAV) that can package an inverted repeat genome that can fold into double-stranded DNA without the need for DNA synthesis or base-pairing between multiple vector genomes (McCarty, (2008) Molec. Therapy 16(10):1648-1656; McCarty et al., (2001) Gene Therapy 8:1248-1254; McCarty et al., (2003) Gene Therapy 10:2112-2118). A limitation of a scAAV vector is that size of the unique transgene, regulatory elements and IRTs to be package in the capsid is about half the size (i.e., ˜2,500 nucleotides of which 2,200 nucleotides may be a transgene and regulatory elements, plus two copies of the ˜145 nucleotide ITRs) of a ssAAV vector genome (i.e., ˜4,900 nucleotides including two ITRs).

scAAV vector genomes are made by using a nucleic acid not comprising the terminal resolution site (TRS), or by altering the TRS, from one rAAV ITR of a vector, e.g, a plasmid, comprising the vector genome thereby preventing initiation of replication from that end (see U.S. Pat. No. 8,784,799). AAV replication within a host cell is initiated at the wild type ITR of the scAAV vector genome and continues through the ITR lacking or comprising an altered terminal resolution site and then back across the genome to create a complementary strand. The resulting complementary single nucleic acid molecule is thus a self-complementary nucleic acid molecule that results in a vector genome with a mutated (is not resolved) ITR in the middle, and wild-type ITRs at each end. In some embodiments, a mutant ITR lacking a TRS or comprising an altered TRS is at the 5′ end of the vector genome. In some embodiments, a mutant ITR lacking a TRS or comprising an altered TRS that is not resolved (cleaved) is at the 3′ end of the vector genome.

Without wishing to be bound by theory, while the two halves of a scAAV genome are complementary, it is unlikely that there is substantial base pairing within the capsid as many of the bases are in contact with amino acid residues of the inner capsid shell and the phosphate backbone is sequestered toward the center (McCarty, Molec. Therapy (2008) 16(10):1648-1656). It likely that upon uncoating, the two halves of the scAAV genome anneal to form a dsDNA hairpin molecule, with a covalently closed ITR at one end and two open-ended ITRs on the other. The ITRs flank a double-stranded region encoding, among other things, the transgene, and regulatory elements in cis thereto.

A viral capsid of an rAAV vector used in the methods described herein may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4^{th} ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. One skilled in the art would know there are likely other AAV variants not yet identified that perform the same or similar function. A full complement of AAV cap proteins includes VP1, VP2, and VP3. The ORF comprising nucleotide sequences encoding AAV VP capsid proteins may comprise less than a full complement of the AAV Cap proteins or the full complement of AAV cap proteins may be provided.

In another embodiment, the present disclosure provides for the use of ancestral AAV vectors for use in therapeutic in vivo gene therapy. Specifically, in silico-derived sequences may be synthesized de novo and characterized for biological activities. Prediction and synthesis of ancestral sequences, in addition to assembly into an rAAV vector, may be accomplished using methods described in WO 2015/054653, the contents of which are incorporated by reference herein. Notably, rAAV vectors assembled from ancestral viral sequences may exhibit reduced susceptibility to pre-existing immunity in human populations as compared to contemporary viruses or portions thereof.

In some embodiments, an rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example, a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or submit can be replaced by a B19 cap protein or subunit. For example, in one embodiment, a VP3 subunit of an AAV capsid can be replaced by a VP2 subunit of B19.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., an rAAV vector genome) are expressed.

In some embodiments, the viral capsid of an rAAV vector used in the methods described herein may be from a wild type AAV or a variant AAV such as AAV1, AAV3a, AAV3b, AAV6 or AAV8.

In some embodiments, an rAAV vector is useful for treating or preventing a disease, disorder or condition. In some embodiments, the disease, disorder or condition includes (but is not limited to) hemophilia (e.g., hemophilia A or B), Duchenne's Muscular Dystrophy (DMD), Wilson's disease, Amyotrophic Lateral Sclerosis (ALS), Hereditary Angioedema (HAE), Pompe Disease, or hypertrophic cardiomyopathy caused by MYBPC3 mutations.

### Methods and Compositions

The present disclosure relates to compositions and methods for enhancing rAAV vector production in baculovirus expression vector (BEV) systems in insect cells. As discussed supra, an inverse correlation was observed between clipped VP1 and VP2 protein levels and the relative in vitro potency, where in vitro potency decreased as the level of clipped VP1 and VP2 proteins increased with time post-infection. This increase in clipped VP1 and VP2 proteins with time post-infection and the concomitant decrease in in vitro potency was observed both at large-scale production (2000 L) and small-scale production (2 L, 10 L or 200 L).

Specifically, two forms of clipped VP1 and VP2 proteins were identified. The first clipped species corresponds to the C-terminal peptide remaining after proteolytic cleavage between VP1 amino acid residues Gly115 and Arg116 of wild-type AAV6. The second clipped species corresponds to the C-terminal peptide remaining after proteolytic cleavage between VP1 and VP2 amino acid residues that correspond to Gly189 and Glu190 of wild-type AAV6.

Accordingly, in some aspects, a method for producing recombinant adeno-associated virus (rAAV) vector is provided. The method comprises contacting an insect cell with one or more recombinant baculovirus(es), each baculovirus comprising a heterologous sequence; and culturing the insect cell under suitable conditions and for a time sufficient to produce rAAV vector with no more than 15% clipping between VP1 amino acid residues Gly115 and Arg116 of wild-type AAV6, or the corresponding amino acids in the VP1 protein of another AAV serotype. Thus, the method produces rAAV vector wherein no more than 15% of the total VP1 protein of the viral capsid is clipped between amino acid residues Gly115 and Arg116.

In some aspects, a method for producing recombinant adeno-associated virus (rAAV) vector is provided. The method comprises contacting an insect cell with one or more recombinant baculovirus(es), each baculovirus comprising a heterologous sequence; and culturing the insect cell under suitable conditions and for a time sufficient to produce rAAV vector with no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype.

Also disclosed herein are methods for increasing in vitro potency of a recombinant adeno-associated viral (AAV) vector. The methods comprise contacting an insect cell with one or more recombinant baculovirus(es), each baculovirus comprising a heterologous sequence; and optimizing the time for culturing the insect cell under suitable conditions such that the in vitro potency of the rAAV vector is between 10%-500% with no more than 15% clipping between amino acid residues 115G and 116R on VP1 protein.

In some aspects, a method for increasing in vitro potency of a recombinant adeno-associated viral (AAV) vector is provided. The method comprises contacting an insect cell with one or more recombinant baculovirus(es), each baculovirus comprising a heterologous sequence; and optimizing the time for culturing the insect cell under suitable conditions such that the in vitro potency of the rAAV vector is between 10%-500% with no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype.

Any viral AAV capsid (e.g., wild type or variant) may be used in the methods described herein, including but not limited to, capsid from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4^{th} ed., Lippincott-Raven Publishers). In some embodiments, the capsid is derived from AAV1, AAV3A, AAV3B, AAV6 and/or AAV8. In some embodiments, the capsid is derived from AAV6.

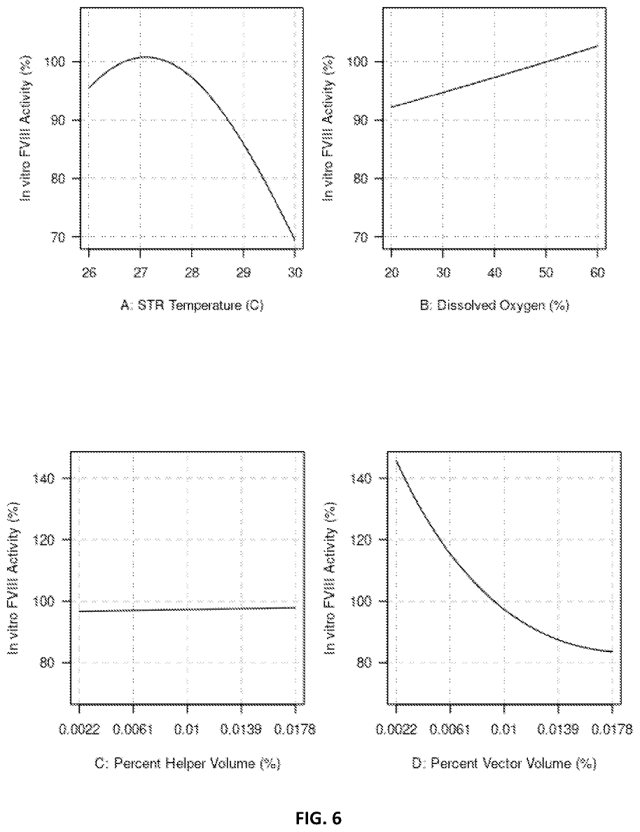

Those skilled in the art can easily ascertain, based on a comparison of the amino acid sequences of capsid proteins (VP1 and VP2) of various AAV serotypes, where “VP1 amino acid residues that correspond to Gly115 and Arg116 of wild-type AAV6” and where “VP1 and VP2 amino acid residues that correspond to Gly189 and Glu190 of wild type AAV6” would be in VP1 and VP2 capsid proteins of any given AAV serotype. See also FIG. 5 for an alignment of wild-type AAV SEQ ID NOS:1-5 which provides amino acid locations between and across the wild-type (naturally occurring) serotypes AAV1 (SEQ ID NO: 1), AAV3A (SEQ ID NO: 2), AAV3B (SEQ ID NO: 3), AAV6 (SEQ ID NO: 4) and AAV8 (SEQ ID NO: 5).

In some embodiments, the rAAV vector produced has no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1% clipping between the VP1 amino acid residues Gly115 and Arg116 of wild-type AAV6, or the corresponding amino acids in the VP1 protein of another AAV serotype, relative to the total amount of VP1 protein. In some embodiments, the rAAV vector produced has between about 12% and about 15% clipping between the VP1 amino acid residues Gly115 and Arg116 of wild-type AAV6, or the corresponding amino acids in the VP1 protein of another AAV serotype, relative to the total amount of VP1 protein. In some embodiments, the rAAV vector produced has less than 5% clipping between the VP1 amino acid residues Gly115 and Arg116 of wild-type AAV6, or the corresponding amino acids in the VP1 protein of another AAV serotype, relative to the total amount of VP1 protein. In some embodiments, the rAAV vector produced has about 1%, about 1.5%, about 2%, about 2.5%, about 3%, about 3.5%, about 4%, about 4.5% or about 5% clipping between the VP1 amino acid residues Gly115 and Arg116 of wild-type AAV6, or the corresponding amino acids in the VP1 protein of another AAV serotype, relative to the total amount of VP1 protein.

In some embodiments, the rAAV vector produced has no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 2%, or no more than 1% clipping between the VP1 and VP2 amino acid residues Gly189 and Glu190 of wild-type AAV6, or the corresponding amino acids in the VP1 and VP2 proteins of another AAV serotype, relative to the total amount of VP1 and VP2 proteins. In some embodiments, the rAAV vector produced has about 30% to about 60% clipping between the VP1 and VP2 amino acid residues Gly189 and Glu190 of wild-type AAV6, or the corresponding amino acids in the VP1 and VP2 proteins of another AAV serotype, relative to the total amount of VP1 and VP2 proteins.

In some embodiments, the rAAV vector produced has no more than 40% between the VP1 and VP2 amino acid residues Gly189 and Glu190 of wild-type AAV6 and no more than 2% clipping between the VP1 amino acid residues Gly115 and Arg116 of wild-type AAV6, or the corresponding amino acids in the VP1 and VP2 proteins of another AAV serotype.

In some embodiments, the rAAV vector produced has no more than 48% between the VP1 and VP2 amino acid residues Gly189 and Glu190 of wild-type AAV6 and no more than 2.5% clipping between the VP1 amino acid residues Gly115 and Arg116 of wild-type AAV6, or the corresponding amino acids in the VP1 and VP2 proteins of another AAV serotype.

In some embodiments, the rAAV vector produced has no more than 52% between the VP1 and VP2 amino acid residues Gly189 and Glu190 of wild-type AAV6 and no more than 11% clipping between the VP1 amino acid residues Gly115 and Arg116 of wild-type AAV6, or the corresponding amino acids in the VP1 and VP2 proteins of another AAV serotype.

In some embodiments, the rAAV vector produced has no more than 47% between the VP1 and VP2 amino acid residues Gly189 and Glu190 of wild-type AAV6 and no more than 9% clipping between the VP1 amino acid residues Gly115 and Arg116 of wild-type AAV6, or the corresponding amino acids in the VP1 and VP2 proteins of another AAV serotype.

In some embodiments, the rAAV vector produced has no more than 43% between the VP1 and VP2 amino acid residues Gly189 and Glu190 of wild-type AAV6 and no more than 9% clipping between the VP1 and VP2 amino acid residues Gly115 and Arg116 of wild-type AAV6, or the corresponding amino acids in the VP1 and VP2 proteins of another AAV serotype.

In some embodiments, the rAAV vector produced has no more than 49% between the VP1 and VP2 amino acid residues Gly189 and Glu190 of wild-type AAV6 and no more than 13% clipping between the VP1 amino acid residues Gly115 and Arg116 of wild-type AAV6, or the corresponding amino acids in the VP1 and VP2 proteins of another AAV serotype.

In some embodiments, the rAAV vector produced has no more than 37% between the VP1 and VP2 amino acid residues Gly189 and Glu190 of wild-type AAV6 and no more than 9% clipping between the VP1 amino acid residues Gly115 and Arg116 of wild-type AAV6, or the corresponding amino acids in the VP1 and VP2 proteins of another AAV serotype.

In some embodiments, the insect cells are cultured for a time sufficient to produce rAAV vector with no more than 75% clipping on VP1 and VP2 proteins (i.e., the total clipping observed for both species is no more than 75%). In some embodiments, the rAAV produced has no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 2%, or no more than 1% clipping on VP1 and VP2 proteins. In some embodiments, the rAAV produced has between about 35% and about 55% total clipping on VP1 and VP2 proteins.

Clipping on VP1 and VP2 proteins can be determined using assays well known in the art, such as but not limited to, capillary gel electrophoresis (CGE), mass spectrometry (including multi-attribute mass spectrometry), and/or Western blot assays. For example, the multi-attribute method (MAM) based on proteolytic digestion followed by liquid chromatography-mass spectrometry (LC-MS) allows simultaneous, site-specific detection and quantification of product quality attributes (see, for example, Zhang et al. MAbs. 2020 January-December; 12(1): 1783062). The method involves sample preparation comprising denaturation, reduction, alkylation, buffer exchange) followed by trypsin digestion. The resulting peptides are then separated using reversed-phase liquid chromatograph (HPLC) with a shallow elution gradient to generate a peptide map. The HPLC is also coupled to a high resolution mass spectrometer to elucidate amino acid modifications. Custom software such as MassAnalyzer can be used to process the data to perform peak detection, retention time alignment, peak identification and quantification.

Similarly, capillary gel electrophoresis can be used to analyze capsid protein purity with greatly improved accuracy, precision and sensitivity (Zhang et al. Capillary Electrophoresis-Sodium Dodecyl Sulfate with Laser-Induced Fluorescence Detection As a Highly Sensitive and Quality Control-Friendly Method for Monitoring Adeno-Associated Virus Capsid Protein Purity. Hum Gene Ther. 2021 Jan. 22).

“Contacting an insect cell with one or more recombinant baculoviruses” comprises introducing the one or more recombinant baculoviruses into sufficient proximity to the insect cell to allow the transduction of the insect cell by the recombinant baculoviruses. The feasibility of insect cells to produce rAAV vectors using recombinant baculoviruses has been demonstrated by Urabe et al. (Hum Gene Ther 13:1935-43(2002)), Kotin et al. (US 2004/0197895) and Kohlbrenner (US 2006/0166363). Any insect cell which allows for replication of AAV and which can be maintained in culture can be used in the methods described herein For example, the cell line used can be from Spodoptera frugiperda such as the Sf) or Sf2l cell lines, drosophila cell lines, or mosquito cell lines, e.g., Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et a, J. Vir. 63:3822-8 (1989); Kajigaya et al., Proc. Nat'l. Acad. Sci. USA 88: 4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,201,059. A preferred cell line is the Spodoptera frugiperda Sf9 cell line.

Methods to produce recombinant baculoviruses (e.g., baculoviruses modified to comprise a heterologous nucleic acid) are well known in the art, such as, but not limited to flashBAC, BACPAK6 or bac-to-bac site-specific transposition system (see, for example, Kitts et al. (1990) Linearization of baculovirus DNA enhances the recovery of recombinant virus expression vectors. Nucleic Acids Res. 11(19), 5667-72; Kitts et al. (1993) A method for producing recombinant baculovirus expression vectors at high frequency. Biotechniques 14, 810; and Anderson et al (1996) New baculovirus expression vectors for the purification of recombinant proteins from insect cells. Focus 17, 53). In some embodiments, the recombinant baculoviruses used in the methods described herein are purified recombinant baculoviruses. In some embodiments, the recombinant baculoviruses used in the methods described herein are recombinant baculovirus infected insect cells (also called BIICs).

The recombinant baculoviruses comprise a heterologous sequence. The heterologous sequence may comprise a nucleic acid sequence encoding AAV Rep proteins (e.g., Rep78/68, Rep 52/40) and/or AAV Cap proteins (e.g., VP1, VP2, VP3). Baculoviruses comprising nucleic acid sequences encoding AAV Rep and/or AAV Cap proteins are termed helper recombinant baculoviruses. The heterologous sequence may also comprise a nucleic acid sequence encoding a gene of interest (e.g., a transgene) flanked by two AAV inverted terminal repeats (ITRs). Baculoviruses comprising nucleic acid sequences encoding such a transgene is termed a vector recombinant baculovirus.

In some embodiments, the insect cell is contacted with three recombinant baculoviruses—
(i) a helper recombinant baculovirus comprising a heterologous sequence encoding AAV Rep proteins (e.g., Rep 78, Rep 68, Rep 52 and/or Rep 40);
(ii) a helper recombinant baculovirus comprising a heterologous sequence encoding AAV capsid (cap) proteins (e.g. AAV VP1, VP2 and/or VP3 proteins); and
(iii) a vector recombinant baculovirus comprising a heterologous sequence encoding a transgene flanked by two AAV inverted terminal repeats (ITRs), one on the 5′ end, and the other on the 3′end.

In some embodiments, the insect cell is contacted with two recombinant baculoviruses—
(i) a helper recombinant baculovirus comprising a heterologous sequence encoding AAV Rep proteins (e.g., Rep 78, Rep 68, Rep 52 and/or Rep 40) and AAV capsid (cap) proteins (e.g. AAV VP1, VP2 and/or VP3 proteins); and
(ii) a vector recombinant baculovirus comprising a heterologous sequence encoding a transgene flanked by two AAV inverted terminal repeats (ITRs), one on the 5′ end, and the other on the 3′end.

As discussed supra, the transgene flanked by ITRs typically encodes a polypeptide of interest, or a gene of interest (“GOI”). Proteins encoded by the transgene include therapeutic proteins, such as but not limited to, blood clotting factor (e.g., Factor XIII, Factor IX, Factor X, Factor VIII, Factor VIIa, or protein C), mini-dystrophin, C1 esterase inhibitor, copper transporting P-type ATPase (ATP7B), copper-zinc superoxide dismutase 1 (SOD1), myosin binding protein C3, apoE2, arginino succinate synthase, acid alpha-glucosidase, β-Glucocerebrosidase, a-galactosidase, CI inhibitor serine protease inhibitor, CFTR (cystic fibrosis transmembrane regulator protein), one or more zinc finger nucleases for genome editing, or donor sequences used as repair templates for genome editing. In some embodiments, the transgene encodes Factor VIII. In some embodiments, the Factor VIII transgene is flanked by AAV2 ITRs. In some embodiments, the transgene comprises SEQ ID NO: 9.

“Culturing the cells under suitable conditions” refers to growing the insect cells in cell culture media and environmental conditions that allow the production of rAAV vector. Examples of suitable conditions include, but are not limited to, temperature at which the cells are cultured, type of cell culture media, viable cell density target at which the recombinant baculovirus(es) are added, amount of dissolved oxygen in the cell culture medium, amount of helper recombinant baculovirus, and/or amount of vector recombinant baculovirus used in the method.

In some embodiments, the insect cells are cultured for a time sufficient to allow production of rAAV vector with no more than 15% clipping between VP1 amino acid residues that correspond to Gly115 and Arg116 of wild-type AAV6 or the corresponding amino acids in the VP1 protein of another AAV serotype. In some embodiments, the insect cells are cultured for a time sufficient to allow production of rAAV vector with no more than 65% clipping between VP1 and VP2 amino acid residues that correspond to Gly189 and Glu190 and no more than 15% clipping between VP1 amino acid residues that correspond to Gly115 and Arg116 of wild-type AAV6 or the corresponding amino acids in the VP1 and VP2 proteins of another AAV serotype.

In some embodiments, time sufficient to allow production of rAAV vector with no more than 15% clipping between VP1 amino acid residues that correspond to Gly115 and Arg116 of wild-type AAV6 or the corresponding amino acids in the VP1 proteins of another AAV serotype, or to allow production of rAAV vector with no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype is 24 hours, 2 days, 3 days, 4 days, 4.1 days, 4.2 days, 4.3 days, 4.4 days, 4.5 days, 4.6 days, 4.7 days, 4.8 days, 4.9 days, 5 days, 5.1 days, 5.2 days, 5.3 days, 5.4 days, 5.5 days, 6 days, 7 days, 8 days, 9 days or 10 days. In some embodiments, the insect cell is cultured for at least 4.1 days but no more than 10 days prior to recovering the rAAV vector from the insect cell. In some embodiments, the insect cell is cultured for about 4 days, 4.1 days, 4.2 days, 4.3 days, 4.4 days, 4.5 days, 4.6 days, 4.7 days, 4.8 days, 4.9 days, 5 days, 5.1 days, 5.2 days, 5.3 days, 5.4 days, or 5.5 days prior to recovering the rAAV vector from the insect cell. In some embodiments, the insect cell is cultured for about 96 hours to about 128 hours prior to recovering the rAAV vector from the insect cell. In some embodiments, the insect cell is cultured for about 103 hours to about 163 hours prior to recovering the rAAV vector from the insect cell. In some embodiments, the insect cell is cultured for about 108±5 hours prior to recovering the rAAV vector from the insect cell.

Time to sufficient to allow production of rAAV vector (also called “time post-infection” or “batch duration post-infection) refers to the time between contacting the recombinant baculoviruses with the insect cell and harvest (i.e., recovery) of the rAAV vector from the insect cell (i.e., the rAAV is recovered from the insect cell by methods well known in the art such as, but not limited to, digestion, filtration, centrifugation, chromatography and/or viral inactivation steps).

“In vitro potency” refers to the measure of the in vitro (i.e., outside a living organism) activity of the rAAV produced by the methods described herein. For example, in vitro potency may be measured in terms of the infectivity of the rAAV vector, transgene expression and/or function of the protein encoded by the transgene. Assays to measure in vitro potency of rAAV vector are well-known in the art and include (but are not limited to) colorimetric assay, a chromogenic assay, an ELISA-based assay, quantitative PCR, and/or Western blot.

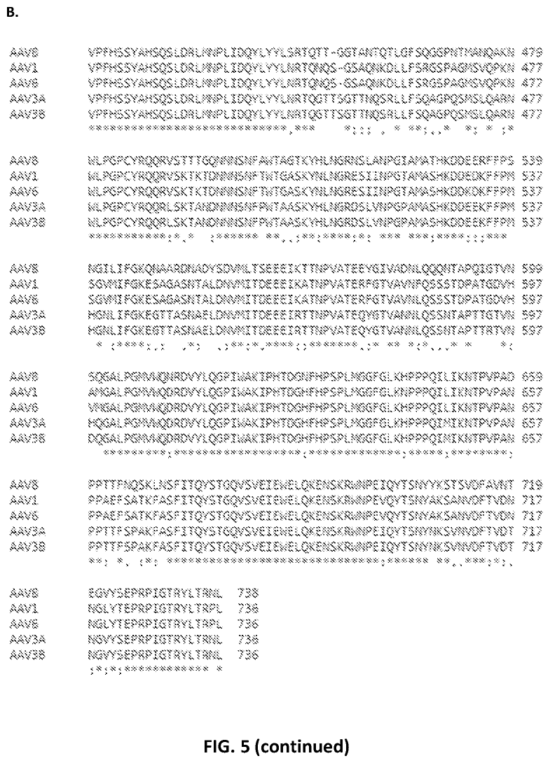

In some embodiments, a colorimetric assay is used to measure the in vitro potency of the rAAV vector. An example of a colorimetric assay to measure the in vitro potency of rAAV vector comprising a transgene encoding blood clotting factor, Factor VIII, involves exposing liver carcinoma cells to AAV, culturing the cells for several days, followed by the use of a chromogenic substrate to quantify Factor VIII activity in harvested media (FIG. 4).

The present disclosure provides methods for increasing (i.e., improving, enhancing, and/or optimizing) the in vitro potency of produced rAAV vector. This increase, improvement, enhancement and/or optimization of in in vitro potency may be measured relative to the in vitro potency value of rAAV vector not produced by the methods described herein. For example, the rAAV vector produced by the methods described herein have no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype, and a concomitant increased, improved or enhanced in vitro potency as compared to rAAV vector having more than 65% clipping between VP1 and VP2 amino acid residues that correspond to Gly189 and Glu190 and more than 15% clipping between VP1 amino acid residues that correspond to Gly115 and Arg116 of wild-type AAV6, or the corresponding amino acids in the VP1 and VP2 proteins of another AAV serotype.

As discussed supra, and in the Examples described herein, the data demonstrated an inverse correlation between the clipped VP1/VP2 protein levels and the relative in vitro potency, where in vitro potency decreased as the level of clipped VP1/VP2 increased with time post-infection. This increase in clipped VP1 and VP2 proteins with time post-infection and the concomitant decrease in in vitro potency was observed both at large-scale production (2000 L) and small-scale production (2 L, 10 L or 200 L). The clipped VP1 and VP2 proteins observed corresponded to two forms—the first clipped species corresponds to the C-terminal peptide remaining after proteolytic cleavage between VP1 amino acid residues Gly115 and Arg116 of wild-type AAV6, while the second clipped species corresponds to the C-terminal peptide remaining after proteolytic cleavage between VP1 and VP2 amino acid residues that correspond to Gly189 and Glu190 of wild-type AAV6.

Literature (Zidori Z, Szelei J, Lacoste M C, Li Y, Gariepy S, Raymond P, Allaire M, Nabi I R, Tijssen P. A viral phospholipase A2 is required for parvovirus infectivity. Dev Cell. 2001; 1(2):291-302; Girod A, Wobus C E, Zidori Z, Ried M, Leike K, Tijssen P, Kleinschmidt J A, Hallek M. The VP1 capsid protein of adeno-associated virus type 2 is carrying a phospholipase A2 domain required for virus infectivity. J Gen Virol. 2002; 83(Pt 5):973-978. doi: 10.1099/0022-1317-83-5-973) has demonstrated that the VP1 protein is critical for the infectivity and in vitro potency of AAV and that the baculovirus expression system naturally produces low levels of VP1 in comparison to wildtype AAV. Therefore, seemingly minor differences in the levels of clipped VP1 proteins may have a significant impact on in vitro potency.

The role of VP1 protein in infectivity was recognized early in the genetic analysis of the AAV genome; mutants lacking the VP1 N-terminal region yielded normal levels of DNA replication and encapsidation, but low viral infectivity (Hermonat P L, et al. Genetics of adeno-associated virus: isolation and preliminary characterization of adeno-associated virus type 2 mutants. J Virol. 1984; 51(2):329-39). The N-terminal regions of VP1 and VP2 are normally internalized in the AAV capsid, forming an electron dense globular structure at the interior face of the 2-fold axis of symmetry (Kronenberg S, et al. A conformational change in the adeno-associated virus type 2 capsid leads to the exposure of hidden VP1 N termini. J Virol. 2005; 79(9):5296-303). In response to heat shock, or acidification associated with trafficking through the endosomal-lysosomal system, these N-terminal extensions are extruded through the pore at the capsid 5-fold axis of symmetry and displayed on the capsid exterior to carry out essential functions in viral infection.

The most prominent feature of the AAV VP1 N-terminal unique region is the phospholipase A2 domain (PLA2), spanning amino acids (aa) 45-103, which is present in all parvoviruses. This phospholipase activity is essential for endosomal escape subsequent to viral attachment and internalization into the target cell (Zidori Z, et al. A viral phospholipase A2 is required for parvovirus infectivity. Dev Cell. 2001; 1(2):291-302; Girod A, et al. The VP1 capsid protein of adeno-associated virus type 2 is carrying a phospholipase A2 domain required for virus infectivity. J Gen Virol. 2002; 83(Pt 5):973-978.).

Capsids with mutations or deletions within the AAV VP1 PLA2 domain retain the ability to attach to target cells and internalize, but are defective for gene expression (Girod A, et al. The VP1 capsid protein of adeno-associated virus type 2 is carrying a phospholipase A2 domain required for virus infectivity. J Gen Virol. 2002; 83(Pt 5):973-978). Cleavage of VP1 proteins at amino acid 115 and/or amino acid 189 of AAV6 would result in loss of the phospholipase A2 domain (PLA2), and consequent loss of infectivity.

In addition to the critical function of the PLA2 domain, three clusters of basic amino acids within the VP1 and VP2 N-terminal regions have been shown to be essential for transport of the capsid into the nucleus (Grieger J C, Snowdy S, Samulski R J. Separate basic region motifs within the adeno-associated virus capsid proteins are essential for infectivity and assembly. J Virol. 2006; 80(11):5199-210). Loss of basic region (BR) 1 (₁₂₀QAKKRVL₁₂₆) (SEQ ID NO: 6) or BR2 (₁₄₀PGKKRPV₁₄₆) (SEQ ID NO: 7) from VP1 reduces AAV infectivity by 4- and 10-fold, respectively. Loss of BR3 (₁₆₆PARKRLN₁₇₂), (SEQ ID NO: 8) which is present in the N-terminal regions of both VP1 and VP2, results in complete loss of vector infectivity. These critical regions are conserved in AAV serotypes 1 to 11 and would be lost from both VP1 and VP2 in the cleaved products resulting from clipping between amino acids 115 and 116 and/or amino acids 189 and 190 of AAV6, and therefore would contribute to reduced in vitro potency.

While loss of the VP1/VP2 N-terminal regions due to cleavage at amino acid 189 is predicted to reduce infectivity and transduction efficiency, it would not be expected to alter cell-type and tissue tropisms, which are governed by surface variable region motifs within the VP3 common region. The primary cellular receptors for most AAV serotypes are glycans, including heparan sulfate, sialic acids or galactose, which bind to VP3 variable regions associated with the capsid 3-fold axis of symmetry (Albright B H, Simon K E, Pillai M, Devlin G W, Asokan A. Modulation of Sialic Acid Dependence Influences the Central Nervous System Transduction Profile of Adeno-associated Viruses. J Virol. 2019; 93(11); Zhang R, Cao L, Cui M, Sun Z, Hu M, Zhang R, Stuart W, Zhao X, Yang Z, Li X, Sun Y, Li S, Ding W, Lou Z, Rao Z. Adeno-associated virus 2 bound to its cellular receptor AAVR. Nat Microbiol. 2019; 4(4):675-682).

The N-terminal regions of VP1 and VP2 remain within the capsid interior at the cellular attachment phase of infection, and do not participate in these interactions. Similarly, the more recently identified secondary receptors that are common to most AAV serotypes, AAVR and GPR108, also bind to external structural elements of the capsid associated with the 3-fold axis of symmetry (Albright B H, Simon K E, Pillai M, Devlin G W, Asokan A. Modulation of Sialic Acid Dependence Influences the Central Nervous System Transduction Profile of Adeno-associated Viruses. J Virol. 2019; 93(11); Zhang R, Cao L, Cui M, Sun Z, Hu M, Zhang R, Stuart W, Zhao X, Yang Z, Li X, Sun Y, Li S, Ding W, Lou Z, Rao Z. Adeno-associated virus 2 bound to its cellular receptor AAVR. Nat Microbiol. 2019; 4(4):675-682; Huang L Y, Patel A, Ng R, Miller E B, Halder S, McKenna R, Asokan A, Agbandje-McKenna M. Characterization of the Adeno-Associated Virus 1 and 6 Sialic Acid Binding Site. J Virol. 2016; 90(11):5219-5230).

The presence or absence of the VP1 and VP2 N-terminal regions, which are structurally disordered compared to the VP3 common regions, generally do not contribute to the structural features of the capsid at the 3-fold axis (Agbandje-McKenna M, Kleinschmidt J. AAV capsid structure and cell interactions. Methods Mol Biol. 2011; 807:47-92).

While clipping at amino acid Gly189/Glu190, has been published in literature as the cleavage site for the baculoviral cathepsin (v-CATH) protease (Galibert L, Savy A, Dickx Y, Bonnin D, Bertin B, Mushimiyimana I, van Oers M M, Merten O W. Origins of truncated supplementary capsid proteins in rAAV8 vectors produced with the baculovirus system. PLoS One. 2018; 13(11):e0207414), clipping at position Gly115/Arg116 was not known. In addition, while Galibert et al (2018) and others (US 2019/0054158) proposed potential solutions to prevent the loss of infectivity of rAAV vectors, there was no recognition that batch duration post-infection (i.e., time post-infection) would have an impact VP1/VP2 clipping and potency.

As discussed supra, in vitro potency may be measured in terms of the infectivity of the rAAV vector, transgene expression and/or function of the protein encoded by the transgene. As such, the in vitro potency may be measured in terms of infectivity of the rAAV vector, transgene expression (determined, for example, by quantitative PCR), or protein function (determined, for example, by Western blot, Elisa-based assay, colorimetric assay or chromogenic assay). In some embodiments, the in vitro potency is determined using a reference standard. For example, the in vitro potency values of an arbitrarily chosen batch of rAAV vector produced by the methods described herein may be designated as reference standard and set as 100% activity. The in vitro potency of other batches of rAAV vector produced by the methods described herein may then be calculated as a percentage relative to this reference standard. In some embodiments, the in vitro potency values of an arbitrarily chosen batch of rAAV vector not produced by the methods described herein may be designated as reference standard and set as 100% activity. The in vitro potency of other batches of rAAV vector produced by the methods described herein may then be calculated as a percentage relative to this reference standard.

In some embodiments, the rAAV vector produced by the methods described herein has an in vitro potency between 10%-500% as compared to a reference standard. In some embodiments, the rAAV vector has an in vitro potency of at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard. In some embodiments, the rAAV vector has an in vitro potency of about 50% to about 150% as compared to a reference standard. In some embodiments, the rAAV vector has an in vitro potency of about 70% to about 130% as compared to a reference standard. In some embodiments, the in vitro potency is determined using the colorimetric assay described above and depicted in FIG. 4.

In some embodiments, the rAAV vector has an in vivo potency of at least 5%, at least 10%, at least 15%, at least 20%, at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard.

“In vivo potency” refers to the measure of the in vivo (i.e., inside a living organism) activity of the rAAV produced by the methods described herein. The in vivo potency may be measured in an animal model. For example, in vivo potency may be measured in terms of the infectivity of the rAAV vector, transgene expression and/or function of the protein encoded by the transgene. Assays to measure in vitro potency of rAAV vector are well-known in the art and include (but are not limited to) colorimetric assay, a chromogenic assay, an ELISA-based assay, quantitative PCR, and/or Western blot.

Typically, the quantity of rAAV vector produced is determined by measuring either the amount of capsid protein or the AAV genome. However, due to the presence of empty capsids that do not contain the entire vector genome, quantification based on viral genome (i.e., the genome titer) are preferable. Clinical dosing of rAAV vector are usually based on vector genome (vg) titer per mL and can be determined using several methods known in the art, for example, but not limited to, dot-blot hybridization (Samulski, R. J., Chang, L. S., and Shenk, T. (1989). Helper-free stocks of recombinant adeno-associated viruses: normal integration does not require viral gene expression. J. Virol. 63, 3822-3828) and Southern blotting (McCarty, M. (1946). Purification and properties of desoxyribonuclease isolated from beef pancreas. J. Gen. Physiol. 29, 123-139), which are not affected by secondary structure of construct terminal regions; UV spectrophotometry (Sommer, J. M., Smith, P. H., Parthasarathy, S., Isaacs, J., Vijay, S., Kieran, J., et al. (2003). Quantification of adeno-associated virus particles and empty capsids by optical density measurement. Mol. Ther. 7, 122-128) and PicoGreen based fluorimetry (Piedra, J., Ontiveros, M., Miravet, S., Penalva, C., Monfar, M., and Chillon, M. (2015). Development of a rapid, robust, and universal picogreen-based method to titer adeno-associated vectors. Hum. Gene Ther. Methods 26, 35-42), ELISA Sondhi, D., Peterson, D. A., Giannaris, E. L., Sanders, C. T., Mendez, B. S., De, B., et al. (2005). AAV2-mediated CLN2 gene transfer to rodent and non-human primate brain results in long-term TPP-I expression compatible with therapy for LINCL. Gene Ther. 12, 1618-1632), and quantitative real-time PCR (qPCR) (D'Costa, S., Blouin, V., Broucque, F., Penaud-Budloo, M., Fçranois, A., Perez, I. C., et al. (2016). Practical utilization of recombinant AAV vector reference standards: focus on vector genomes titration by free ITR qPCR. Mol. Ther. Methods Clin. Dev. 3:16019).

In some embodiments, the genome titer measured prior to recovery of rAAV vector from the insect cell is at least 1×10¹⁰ viral genomes (vg)/ml. In some embodiments, the genome titer measured prior to recovery of rAAV vector from the insect cell is at least 1×10¹⁰·2×10¹⁰, 3×10¹⁰, 4×10¹⁰, 5×10¹⁰, 6×10¹⁰, 7×10¹⁰, 8×10¹⁰, 9×10¹⁰, or 1×10¹¹ viral genomes (vg)/ml. In some embodiments, the genome titer measured prior to recovery of rAAV vector from the insect cell is at least 8×10¹⁰ viral genomes (vg)/ml.

In some embodiments, the genome titer measured after recovery of rAAV vector from the insect cell is at least 5×10⁹ viral genomes (vg)/ml. In some embodiments, the genome titer measured after recovery of rAAV vector from the insect cell is at least 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ viral genomes (vg)/ml. In some embodiments, the genome titer measured after recovery of rAAV vector from the insect cell is at least 4×10¹⁰ viral genomes (vg)/ml.

In some embodiments, the genome titer is measured by quantitative polymerase chain reaction (qPCR).

In the methods described herein, the insect cells are cultured under suitable conditions and time that allow the production of rAAV vector with no more than 15% clipping between amino acid residues 115G and 116R on VP1 proteins or rAAV vector with no more than 65% clipping between amino acid residues 189G and 190E and no more than 15% clipping between amino acid residues 115G and 116R on VP1 and VP2 proteins of wild-type AAV6, or the corresponding amino acids in the VP1 and VP2 proteins of another AAV serotype. As discussed supra, suitable conditions include but are not limited to, temperature at which the cells are cultured, type of cell culture media, viable cell density target at which the recombinant baculovirus(es) are added, amount of dissolved oxygen in the culture medium, amount of helper recombinant baculovirus, and/or amount of vector recombinant baculovirus used in the method.

In particular, it was determined that the temperature at which the insect cell is cultured, the amount of vector recombinant baculovirus and amount of dissolved oxygen had a significant impact on in vitro potency. Generally, the temperature at which the insect cell is cultured and the amount of vector recombinant baculovirus showed an inverse correlation to the relative in vitro potency, where in vitro potency decreased as the culture temperature and the amount of vector recombinant baculoviruses increased. The amount of dissolved oxygen appeared to have a positive correlation with in vitro potency, i.e., the in vitro potency increased as the amount of dissolved oxygen increased.

In some embodiments, the insect cell is cultured at a temperature lower than 37° C. In some embodiments, the insect cell is cultured at a temperature below 30° C. In some embodiments, the insect cell is cultured at a temperature between 26° C. and 30° C. In some embodiments, the insect cell is cultured at a temperature between 27° C. and 29° C. In some embodiments, the insect cell is cultured at a temperature of about 25° C., about 26° C., about 27° C., about 28° C., about 29° C., about 30° C. or about 31° C. In some embodiments, the insect cell is cultured at a temperature of about 27-28° C. In some embodiments, the insect cell is cultured at a temperature of about 28° C.

In some embodiments, the amount of helper recombinant baculovirus contacted with the insect cell is between about 0.0022 to about 0.0178% volume relative to the total culture volume. In some embodiments, the amount of helper recombinant baculovirus contacted with the insect cell is about 0.01% volume relative to the total culture volume.

In some embodiments, the amount of vector recombinant baculovirus contacted with the insect cell is between about 0.0022 to about 0.0178% volume relative to the total culture volume. In some embodiments, the amount of vector recombinant baculovirus contacted with the insect cell is about 0.01% volume relative to the total culture volume.

In some embodiments, the helper recombinant baculovirus and/or the vector recombinant baculovirus are in the form of recombinant baculovirus infected insect cells (BIICs). In some embodiments, the helper recombinant baculovirus and/or the vector recombinant baculovirus are purified (i.e., the recombinant baculovirus are isolated from the insect cells).

In some embodiments, the amount of dissolved oxygen in the culture medium is about 20% to about 100% of air saturation. Dissolved oxygen may be measured by a fluorescent sensor, an optical probe, or a polarographic probe and is expressed as percentage of air saturation. In some embodiments, the amount of dissolved oxygen in the culture medium is about 20%, about 30%, about 40%, about 50%, about 60%, about 70%, about 80%, about 90% or about 100%. In some embodiments, the amount of dissolved oxygen in the culture medium is between about 40% to 50% of air saturation.

In some embodiments, the amount of vector recombinant baculovirus contacted with the insect cell is about 0.01% volume relative to the total culture volume. The amount of helper recombinant baculovirus contacted with the insect cell is about 0.01% volume relative to the total culture volume. The amount of dissolved oxygen in the culture medium is between about 40% to 50% of air saturation. The insect cells are cultured at a temperature of about 26-28° C. for about 108±5 hours prior to recovering the rAAV vector from the insect cell.

The insect cell may be cultured in any appropriate cell culture medium, such as, for example, Sf-900 III SFM (ThermoFisher), ExpiSf CD Medium (ThermoFisher) and Grace's medium (Sigma Aldrich). The insect cells may be cultured in a volume of less than 2 L, at least 2 L, at least 10 L, at least 250 L, or at least 2000 L.

Any insect cell capable of producing rAAV vector can be used in the methods described herein. In some embodiments, the insect cell is Spodoptera frugiperda, such as the Sf9 or Sf21 cell lines, drosophila cell lines, or mosquito cell lines, e.g., Aedes albopictus derived cell lines. A preferred cell line is the Spodoptera frugiperda Sf9 cell line.

The insect cells may be grown in suspension culture or may be adherent. In some embodiments, the insect cells are grown or maintained in a serum-free culture medium. In some embodiments, the insect cells are grown or maintained in roller bottles or expanded roller bottles. In some embodiments, the insect cells are grown in bioreactors. In some embodiments, the insect cells are grown in bags or flasks. In some embodiments, the insect cells are grown in a WAVE bioreactor. In some embodiments, the insect cells are grown in a stirred tank bioreactor.

As discussed supra, rAAV vector of any wild type or variant serotype may be produced by the methods described herein. Examples of rAAV vector produced by the methods described herein include, but are not limited to, rAAV1, rAAV2, rAAV3, rAAV3A, rAAV3B, rAAV4, rAAV5, rAAV6, rAAV7, rAAV8, rAAV9, rAAV10, rAAVrh10, rAAVrh74, rAAV12, rAAV2i8, rAAV1.1, rAAV2.5, rAAV6.1, rAAV6.3.1, rAAV9.45, rAAV hu.26, rAAV1.1, rAAV2.5, rAAV6.1, rAAV6.3.1, rAAV2i8, rAAV29G, rAVV-LK03, rAAV2-TT, rAAV2-TT-S312N, and rAAV3B-S312N. In some embodiments, the rAAV vector is rAAV1, rAAV3A, rAAV3B, rAAV6 and/or rAAV8. In some embodiments, the rAAV vector is rAAV6.

In some embodiments, the rAAV vector comprises PF-07055480 (also called “SB-525” herein). PF-07055480/SB-525 vector is a rAAV2/6 vector comprising an AAV6 capsid and AAV2 ITR sequences flanking a wild-type or mutated Serpin1 enhancer linked to a TTRm promoter operably linked to a polynucleotide encoding FVIII (e.g., SEQ ID NO:9). Exemplary vector sequences are described in for example, WO 2017/074526.

Exemplary AAV2 5′ ITR is:

(SEQ ID NO: 10)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT.

Exemplary AAV2 3′ITR is:

(SEQ ID NO: 11)
AGGAACCCCTAGTGATGGAGTTGGCCACTCCCTCTCTGCGCGCTCGCTCG
CTCACTGAGGCCGCCCGGGCTTTGCCCGGGCGGCCTCAGTGAGCGAGCGA
GCGCGCAG.

SEQ ID NO:12 displays the human FVIII amino acid sequence with signal peptide:

MQIELSTCFFLCLLRFCFSATRRYYLGAVELSWDYMQSDLGELPVDARFP
PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVFDEGKSWHSETKNSLMQDRDAASARAWPKM
HTVNGYVNRSLPGLIGCHRKSVYWHVIGMGTTPEVHSIFLEGHTFLVRNH
RQASLEISPITFLTAQTLLMDLGQFLLFCHISSHQHDGMEAYVKVDSCPE
EPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNPPVLKRHQREITRTTLQSDQEEIDYDDTISVEMKKE
DFDIYDEDENQSPRSFQKKTRHYFIAAVERLWDYGMSSSPHVLRNRAQSG
SVPQFKKVVFQEFTDGSFTQPLYRGELNEHLGLLGPYIRAEVEDNIMVTF
RNQASRPYSFYSSLISYEEDQRQGAEPRKNFVKPNETKTYFWKVQHHMAP
TKDEFDCKAWAYFSDVDLEKDVHSGLIGPLLVCHTNTLNPAHGRQVTVQE
FALFFTIFDETKSWYFTENMERNCRAPCNIQMEDPTFKENYRFHAINGYI
MDTLPGLVMAQDQRIRWYLLSMGSNENIHSIHFSGHVFTVRKKEEYKMAL
YNLYPGVFETVEMLPSKAGIWRVECLIGEHLHAGMSTLFLVYSNKCQTPL
GMASGHIRDFQITASGQYGQWAPKLARLHYSGSINAWSTKEPFSWIKVDL
LAPMIIHGIKTQGARQKFSSLYISQFIIMYSLDGKKWQTYRGNSTGTLMV
FFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNN
PKEWLQVDFQKTMKVTGVTTQGVKSLLTSMYVKEFLISSSQDGHQWTLFF
QNGKVKVFQGNQDSFTPVVNSLDPPLLTRYLRIHPQSWVHQIALRMEVLG
CEAQDLY.

The signal peptide portion of SEQ ID NO:12 is MQIELSTCFFLCLLRFCFS (SEQ ID NO:13), which is cleaved off when the protein is secreted.

In some embodiments, an exemplary SERPIN1 enhancer is

(SEQ ID NO: 14)
GGGGGAGGCTGCTGGTGAATATTAACCAAGATCACCCCAGTTACCGGAGG
AGCAAACAGGGACTAAGTTCACACGCGTGGTACC.

An exemplary TTRm promoter is

(SEQ ID NO: 15)
GTCTGTCTGCACATTTCGTAGAGCGAGTGTTCCGATACTCTAATCTCCCT
AGGCAAGGTTCATATTTGTGTAGGTTACTTATTCTCCTTTTGTTGACTAA
GTCAATAATCAGAATCAGCAGGTTTGGAGTCAGCTTGGCAGGGATCAGCA
GCCTGGGTTGGAAGGAGGGGGTATAAAAGCCCCTTCACCAGGAGAAGCCG
TCACACAGATCCACAAGCTCCTG.

An exemplary coding sequence for FVIII is:

(SEQ ID NO: 16)
ATGCAGATCGAGCTCTCCACCTGCTTCTTTCTGTGCCTGTTGAGATTCTG
CTTCAGCGCCACCAGGAGATACTACCTGGGGGCTGTGGAGCTGAGCTGGG
ACTACATGCAGTCTGACCTGGGGGAGCTGCCTGTGGATGCCAGGTTCCCC
CCCAGAGTGCCCAAGAGCTTCCCCTTCAACACCTCTGTGGTGTACAAGAA
GACCCTGTTTGTGGAGTTCACTGACCACCTGTTCAACATTGCCAAGCCCA
GGCCCCCCTGGATGGGCCTGCTGGGCCCCACCATCCAGGCTGAGGTGTAT
GACACTGTGGTGATCACCCTGAAGAACATGGCCAGCCACCCTGTGAGCCT
GCATGCTGTGGGGGTGAGCTACTGGAAGGCCTCTGAGGGGGCTGAGTATG
ATGACCAGACCAGCCAGAGGGAGAAGGAGGATGACAAGGTGTTCCCTGGG
GGCAGCCACACCTATGTGTGGCAGGTGCTGAAGGAGAATGGCCCCATGGC
CTCTGACCCCCTGTGCCTGACCTACAGCTACCTGAGCCATGTGGACCTGG
TGAAGGACCTGAACTCTGGCCTGATTGGGGCCCTGCTGGTGTGCAGGGAG
GGCAGCCTGGCCAAGGAGAAGACCCAGACCCTGCACAAGTTCATCCTGCT
GTTTGCTGTGTTTGATGAGGGCAAGAGCTGGCACTCTGAAACCAAGAACA
GCCTGATGCAGGACAGGGATGCTGCCTCTGCCAGGGCCTGGCCCAAGATG
CACACTGTGAATGGCTATGTGAACAGGAGCCTGCCTGGCCTGATTGGCTG
CCACAGGAAGTCTGTGTACTGGCATGTGATTGGCATGGGCACCACCCCTG
AGGTGCACAGCATCTTCCTGGAGGGCCACACCTTCCTGGTCAGGAACCAC
AGGCAGGCCAGCCTGGAGATCAGCCCCATCACCTTCCTGACTGCCCAGAC
CCTGCTGATGGACCTGGGCCAGTTCCTGCTGTTCTGCCACATCAGCAGCC
ACCAGCATGATGGCATGGAGGCCTATGTGAAGGTGGACAGCTGCCCTGAG
GAGCCCCAGCTGAGGATGAAGAACAATGAGGAGGCTGAGGACTATGATGA
TGACCTGACTGACTCTGAGATGGATGTGGTGAGGTTTGATGATGACAACA
GCCCCAGCTTCATCCAGATCAGGTCTGTGGCCAAGAAGCACCCCAAGACC
TGGGTGCACTACATTGCTGCTGAGGAGGAGGACTGGGACTATGCCCCCCT
GGTGCTGGCCCCTGATGACAGGAGCTACAAGAGCCAGTACCTGAACAATG
GCCCCCAGAGGATTGGCAGGAAGTACAAGAAGGTCAGGTTCATGGCCTAC
ACTGATGAAACCTTCAAGACCAGGGAGGCCATCCAGCATGAGTCTGGCAT
CCTGGGCCCCCTGCTGTATGGGGAGGTGGGGGACACCCTGCTGATCATCT
TCAAGAACCAGGCCAGCAGGCCCTACAACATCTACCCCCATGGCATCACT
GATGTGAGGCCCCTGTACAGCAGGAGGCTGCCCAAGGGGGTGAAGCACCT
GAAGGACTTCCCCATCCTGCCTGGGGAGATCTTCAAGTACAAGTGGACTG
TGACTGTGGAGGATGGCCCCACCAAGTCTGACCCCAGGTGCCTGACCAGA
TACTACAGCAGCTTTGTGAACATGGAGAGGGACCTGGCCTCTGGCCTGAT
TGGCCCCCTGCTGATCTGCTACAAGGAGTCTGTGGACCAGAGGGGCAACC
AGATCATGTCTGACAAGAGGAATGTGATCCTGTTCTCTGTGTTTGATGAG
AACAGGAGCTGGTACCTGACTGAGAACATCCAGAGGTTCCTGCCCAACCC
TGCTGGGGTGCAGCTGGAGGACCCTGAGTTCCAGGCCAGCAACATCATGC
ACAGCATCAATGGCTATGTGTTTGACAGCCTGCAGCTGTCTGTGTGCCTG
CATGAGGTGGCCTACTGGTACATCCTGAGCATTGGGGCCCAGACTGACTT
CCTGTCTGTGTTCTTCTCTGGCTACACCTTCAAGCACAAGATGGTGTATG
AGGACACCCTGACCCTGTTCCCCTTCTCTGGGGAGACTGTGTTCATGAGC
ATGGAGAACCCTGGCCTGTGGATTCTGGGCTGCCACAACTCTGACTTCAG
GAACAGGGGCATGACTGCCCTGCTGAAAGTCTCCAGCTGTGACAAGAACA
CTGGGGACTACTATGAGGACAGCTATGAGGACATCTCTGCCTACCTGCTG
AGCAAGAACAATGCCATTGAGCCCAGGAGCTTCAGCCAGAATCCACCCGT
CCTTAAGCGCCATCAGCGCGAGATCACCAGGACCACCCTGCAGTCTGACC
AGGAGGAGATTGACTATGATGACACCATCTCTGTGGAGATGAAGAAGGAG
GACTTTGACATCTACGACGAGGACGAGAACCAGAGCCCCAGGAGCTTCCA
GAAGAAGACCAGGCACTACTTCATTGCTGCTGTGGAGAGGCTGTGGGACT
ATGGCATGAGCAGCAGCCCCCATGTGCTGAGGAACAGGGCCCAGTCTGGC
TCTGTGCCCCAGTTCAAGAAGGTGGTGTTCCAGGAGTTCACTGATGGCAG
CTTCACCCAGCCCCTGTACAGAGGGGAGCTGAATGAGCACCTGGGCCTGC
TGGGCCCCTACATCAGGGCTGAGGTGGAGGACAACATCATGGTGACCTTC
AGGAACCAGGCCAGCAGGCCCTACAGCTTCTACAGCAGCCTGATCAGCTA
TGAGGAGGACCAGAGGCAGGGGGCTGAGCCCAGGAAGAACTTTGTGAAGC
CCAATGAAACCAAGACCTACTTCTGGAAGGTGCAGCACCACATGGCCCCC
ACCAAGGATGAGTTTGACTGCAAGGCCTGGGCCTACTTCTCTGATGTGGA
CCTGGAGAAGGATGTGCACTCTGGCCTGATTGGCCCCCTGCTGGTGTGCC
ACACCAACACCCTGAACCCTGCCCATGGCAGGCAGGTGACTGTGCAGGAG
TTTGCCCTGTTCTTCACCATCTTTGATGAAACCAAGAGCTGGTACTTCAC
TGAGAACATGGAGAGGAACTGCAGGGCCCCCTGCAACATCCAGATGGAGG
ACCCCACCTTCAAGGAGAACTACAGGTTCCATGCCATCAATGGCTACATC
ATGGACACCCTGCCTGGCCTGGTGATGGCCCAGGACCAGAGGATCAGGTG
GTACCTGCTGAGCATGGGCAGCAATGAGAACATCCACAGCATCCACTTCT
CTGGCCATGTGTTCACTGTGAGGAAGAAGGAGGAGTACAAGATGGCCCTG
TACAACCTGTACCCTGGGGTGTTTGAGACTGTGGAGATGCTGCCCAGCAA
GGCTGGCATCTGGAGGGTGGAGTGCCTGATTGGGGAGCACCTGCATGCTG
GCATGAGCACCCTGTTCCTGGTGTACAGCAACAAGTGCCAGACCCCCCTG
GGCATGGCCTCTGGCCACATCAGGGACTTCCAGATCACTGCCTCTGGCCA
GTATGGCCAGTGGGCCCCCAAGCTGGCCAGGCTGCACTACTCTGGCAGCA
TCAATGCCTGGAGCACCAAGGAGCCCTTCAGCTGGATCAAGGTGGACCTG
CTGGCCCCCATGATCATCCATGGCATCAAGACCCAGGGGGCCAGGCAGAA
GTTCAGCAGCCTGTACATCAGCCAGTTCATCATCATGTACAGCCTGGATG
GCAAGAAGTGGCAGACCTACAGGGGCAACAGCACTGGCACCCTGATGGTG
TTCTTTGGCAATGTGGACAGCTCTGGCATCAAGCACAACATCTTCAACCC
CCCCATCATTGCCAGATACATCAGGCTGCACCCCACCCACTACAGCATCA
GGAGCACCCTGAGGATGGAGCTGATGGGCTGTGACCTGAACAGCTGCAGC
ATGCCCCTGGGCATGGAGAGCAAGGCCATCTCTGATGCCCAGATCACTGC
CAGCAGCTACTTCACCAACATGTTTGCCACCTGGAGCCCCAGCAAGGCCA
GGCTGCATCTGCAGGGCAGGAGCAATGCCTGGAGGCCCCAGGTCAACAAC
CCCAAGGAGTGGCTGCAGGTGGACTTCCAGAAGACCATGAAGGTGACTGG
GGTGACCACCCAGGGGGTGAAGAGCCTGCTGACCAGCATGTATGTGAAGG
AGTTCCTGATCAGCAGCAGCCAGGATGGCCACCAGTGGACCCTGTTCTTC
CAGAATGGCAAGGTGAAGGTGTTCCAGGGCAACCAGGACAGCTTCACCCC
TGTGGTGAACAGCCTGGACCCCCCCCTGCTGACCAGATACCTGAGGATTC
ACCCCCAGAGCTGGGTGCACCAGATTGCCCTGAGGATGGAGGTGCTGGGC
TGTGAGGCCCAGGACCTGTACTGA.

An exemplary sequence for the expression cassette flanked by the inverted terminal repeat sequences is:

(SEQ ID NO: 9)
GCGGCCTAAGCTTGGAACCATTGCCACCTTCAGGGGGAGGCTGCTGGTGA - 50
ATATTAACCAAGATCACCCCAGTTACCGGAGGAGCAAACAGGGACTAAGT - 100
TCACACGCGTGGTACCGTCTGTCTGCACATTTCGTAGAGCGAGTGTTCCG - 150
ATACTCTAATCTCCCTAGGCAAGGTTCATATTTGTGTAGGTTACTTATTC - 200
TCCTTTTGTTGACTAAGTCAATAATCAGAATCAGCAGGTTTGGAGTCAGC - 250
TTGGCAGGGATCAGCAGCCTGGGTTGGAAGGAGGGGGTATAAAAGCCCCT - 300
TCACCAGGAGAAGCCGTCACACAGATCCACAAGCTCCTGAAGAGGTAAGG - 350
GTTTAAGTTATCGTTAGTTCGTGCACCATTAATGTTTAATTACCTGGAGC - 400
ACCTGCCTGAAATCATTTTTTTTTCAGGTTGGCTAGTATGCAGATCGAGC - 450
TCTCCACCTGCTTCTTTCTGTGCCTGTTGAGATTCTGCTTCAGCGCCACC - 500
AGGAGATACTACCTGGGGGCTGTGGAGCTGAGCTGGGACTACATGCAGTC - 550
TGACCTGGGGGAGCTGCCTGTGGATGCCAGGTTCCCCCCCAGAGTGCCCA - 600
AGAGCTTCCCCTTCAACACCTCTGTGGTGTACAAGAAGACCCTGTTTGTG - 650
GAGTTCACTGACCACCTGTTCAACATTGCCAAGCCCAGGCCCCCCTGGAT - 700
GGGCCTGCTGGGCCCCACCATCCAGGCTGAGGTGTATGACACTGTGGTGA - 750
TCACCCTGAAGAACATGGCCAGCCACCCTGTGAGCCTGCATGCTGTGGGG - 800
GTGAGCTACTGGAAGGCCTCTGAGGGGGCTGAGTATGATGACCAGACCAG - 850
CCAGAGGGAGAAGGAGGATGACAAGGTGTTCCCTGGGGGCAGCCACACCT - 900
ATGTGTGGCAGGTGCTGAAGGAGAATGGCCCCATGGCCTCTGACCCCCTG - 950
TGCCTGACCTACAGCTACCTGAGCCATGTGGACCTGGTGAAGGACCTGAA - 1000
CTCTGGCCTGATTGGGGCCCTGCTGGTGTGCAGGGAGGGCAGCCTGGCCA - 1050
AGGAGAAGACCCAGACCCTGCACAAGTTCATCCTGCTGTTTGCTGTGTTT - 1100
GATGAGGGCAAGAGCTGGCACTCTGAAACCAAGAACAGCCTGATGCAGGA - 1150
CAGGGATGCTGCCTCTGCCAGGGCCTGGCCCAAGATGCACACTGTGAATG - 1200
GCTATGTGAACAGGAGCCTGCCTGGCCTGATTGGCTGCCACAGGAAGTCT - 1250
GTGTACTGGCATGTGATTGGCATGGGCACCACCCCTGAGGTGCACAGCAT - 1300
CTTCCTGGAGGGCCACACCTTCCTGGTCAGGAACCACAGGCAGGCCAGCC - 1350
TGGAGATCAGCCCCATCACCTTCCTGACTGCCCAGACCCTGCTGATGGAC - 1400
CTGGGCCAGTTCCTGCTGTTCTGCCACATCAGCAGCCACCAGCATGATGG - 1450
CATGGAGGCCTATGTGAAGGTGGACAGCTGCCCTGAGGAGCCCCAGCTGA - 1500
GGATGAAGAACAATGAGGAGGCTGAGGACTATGATGATGACCTGACTGAC - 1550
TCTGAGATGGATGTGGTGAGGTTTGATGATGACAACAGCCCCAGCTTCAT - 1600
CCAGATCAGGTCTGTGGCCAAGAAGCACCCCAAGACCTGGGTGCACTACA - 1650
TTGCTGCTGAGGAGGAGGACTGGGACTATGCCCCCCTGGTGCTGGCCCCT - 1700
GATGACAGGAGCTACAAGAGCCAGTACCTGAACAATGGCCCCCAGAGGAT - 1750
TGGCAGGAAGTACAAGAAGGTCAGGTTCATGGCCTACACTGATGAAACCT - 1800
TCAAGACCAGGGAGGCCATCCAGCATGAGTCTGGCATCCTGGGCCCCCTG - 1850
CTGTATGGGGAGGTGGGGGACACCCTGCTGATCATCTTCAAGAACCAGGC - 1900
CAGCAGGCCCTACAACATCTACCCCCATGGCATCACTGATGTGAGGCCCC - 1950
TGTACAGCAGGAGGCTGCCCAAGGGGGTGAAGCACCTGAAGGACTTCCCC - 2000
ATCCTGCCTGGGGAGATCTTCAAGTACAAGTGGACTGTGACTGTGGAGGA - 2050
TGGCCCCACCAAGTCTGACCCCAGGTGCCTGACCAGATACTACAGCAGCT - 2100
TTGTGAACATGGAGAGGGACCTGGCCTCTGGCCTGATTGGCCCCCTGCTG - 2150
ATCTGCTACAAGGAGTCTGTGGACCAGAGGGGCAACCAGATCATGTCTGA - 2200
CAAGAGGAATGTGATCCTGTTCTCTGTGTTTGATGAGAACAGGAGCTGGT - 2250
ACCTGACTGAGAACATCCAGAGGTTCCTGCCCAACCCTGCTGGGGTGCAG - 2300
CTGGAGGACCCTGAGTTCCAGGCCAGCAACATCATGCACAGCATCAATGG - 2350
CTATGTGTTTGACAGCCTGCAGCTGTCTGTGTGCCTGCATGAGGTGGCCT - 2400
ACTGGTACATCCTGAGCATTGGGGCCCAGACTGACTTCCTGTCTGTGTTC - 2450
TTCTCTGGCTACACCTTCAAGCACAAGATGGTGTATGAGGACACCCTGAC - 2500
CCTGTTCCCCTTCTCTGGGGAGACTGTGTTCATGAGCATGGAGAACCCTG - 2550
GCCTGTGGATTCTGGGCTGCCACAACTCTGACTTCAGGAACAGGGGCATG - 2600
ACTGCCCTGCTGAAAGTCTCCAGCTGTGACAAGAACACTGGGGACTACTA - 2650
TGAGGACAGCTATGAGGACATCTCTGCCTACCTGCTGAGCAAGAACAATG - 2700
CCATTGAGCCCAGGAGCTTCAGCCAGAATCCACCCGTCCTTAAGCGCCAT - 2750
CAGCGCGAGATCACCAGGACCACCCTGCAGTCTGACCAGGAGGAGATTGA - 2800
CTATGATGACACCATCTCTGTGGAGATGAAGAAGGAGGACTTTGACATCT - 2850
ACGACGAGGACGAGAACCAGAGCCCCAGGAGCTTCCAGAAGAAGACCAGG - 2900
CACTACTTCATTGCTGCTGTGGAGAGGCTGTGGGACTATGGCATGAGCAG - 2950
CAGCCCCCATGTGCTGAGGAACAGGGCCCAGTCTGGCTCTGTGCCCCAGT - 3000
TCAAGAAGGTGGTGTTCCAGGAGTTCACTGATGGCAGCTTCACCCAGCCC - 3050
CTGTACAGAGGGGAGCTGAATGAGCACCTGGGCCTGCTGGGCCCCTACAT - 3100
CAGGGCTGAGGTGGAGGACAACATCATGGTGACCTTCAGGAACCAGGCCA - 3150
GCAGGCCCTACAGCTTCTACAGCAGCCTGATCAGCTATGAGGAGGACCAG - 3200
AGGCAGGGGGCTGAGCCCAGGAAGAACTTTGTGAAGCCCAATGAAACCAA - 3250
GACCTACTTCTGGAAGGTGCAGCACCACATGGCCCCCACCAAGGATGAGT - 3300
TTGACTGCAAGGCCTGGGCCTACTTCTCTGATGTGGACCTGGAGAAGGAT - 3350
GTGCACTCTGGCCTGATTGGCCCCCTGCTGGTGTGCCACACCAACACCCT - 3400
GAACCCTGCCCATGGCAGGCAGGTGACTGTGCAGGAGTTTGCCCTGTTCT - 3450
TCACCATCTTTGATGAAACCAAGAGCTGGTACTTCACTGAGAACATGGAG - 3500
AGGAACTGCAGGGCCCCCTGCAACATCCAGATGGAGGACCCCACCTTCAA - 3550
GGAGAACTACAGGTTCCATGCCATCAATGGCTACATCATGGACACCCTGC - 3600
CTGGCCTGGTGATGGCCCAGGACCAGAGGATCAGGTGGTACCTGCTGAGC - 3650
ATGGGCAGCAATGAGAACATCCACAGCATCCACTTCTCTGGCCATGTGTT - 3700
CACTGTGAGGAAGAAGGAGGAGTACAAGATGGCCCTGTACAACCTGTACC - 3750
CTGGGGTGTTTGAGACTGTGGAGATGCTGCCCAGCAAGGCTGGCATCTGG - 3800
AGGGTGGAGTGCCTGATTGGGGAGCACCTGCATGCTGGCATGAGCACCCT - 3850
GTTCCTGGTGTACAGCAACAAGTGCCAGACCCCCCTGGGCATGGCCTCTG - 3900
GCCACATCAGGGACTTCCAGATCACTGCCTCTGGCCAGTATGGCCAGTGG - 3950
GCCCCCAAGCTGGCCAGGCTGCACTACTCTGGCAGCATCAATGCCTGGAG - 4000
CACCAAGGAGCCCTTCAGCTGGATCAAGGTGGACCTGCTGGCCCCCATGA - 4050
TCATCCATGGCATCAAGACCCAGGGGGCCAGGCAGAAGTTCAGCAGCCTG - 4100
TACATCAGCCAGTTCATCATCATGTACAGCCTGGATGGCAAGAAGTGGCA - 4150
GACCTACAGGGGCAACAGCACTGGCACCCTGATGGTGTTCTTTGGCAATG - 4200
TGGACAGCTCTGGCATCAAGCACAACATCTTCAACCCCCCCATCATTGCC - 4250
AGATACATCAGGCTGCACCCCACCCACTACAGCATCAGGAGCACCCTGAG - 4300
GATGGAGCTGATGGGCTGTGACCTGAACAGCTGCAGCATGCCCCTGGGCA - 4350
TGGAGAGCAAGGCCATCTCTGATGCCCAGATCACTGCCAGCAGCTACTTC - 4400
ACCAACATGTTTGCCACCTGGAGCCCCAGCAAGGCCAGGCTGCATCTGCA - 4450
GGGCAGGAGCAATGCCTGGAGGCCCCAGGTCAACAACCCCAAGGAGTGGC - 4500
TGCAGGTGGACTTCCAGAAGACCATGAAGGTGACTGGGGTGACCACCCAG - 4550
GGGGTGAAGAGCCTGCTGACCAGCATGTATGTGAAGGAGTTCCTGATCAG - 4600
CAGCAGCCAGGATGGCCACCAGTGGACCCTGTTCTTCCAGAATGGCAAGG - 4650
TGAAGGTGTTCCAGGGCAACCAGGACAGCTTCACCCCTGTGGTGAACAGC - 4700
CTGGACCCCCCCCTGCTGACCAGATACCTGAGGATTCACCCCCAGAGCTG - 4750
GGTGCACCAGATTGCCCTGAGGATGGAGGTGCTGGGCTGTGAGGCCCAGG - 4800
ACCTGTACTGAGGATCCAATAAAATATCTTTATTTTCATTACATCTGTGT - 4850
GTTGGTTTTTTGTGTGTTTTCCTGTAACGATCGGGCTCGAGCGC.

SEQ ID NO:9 comprises (from 5′ to 3′) insulator (spacer) sequence Ins1 (nt 14-32 of SEQ ID NO:9), Serpin1 enhancer CRMSBS2 (nt 33-104 of SEQ ID NO:9), transthyretin minimal promoter TTRm (nt 117-339 of SEQ ID NO:9), SBR Intron3 (nt 340-432 of SEQ ID NO:9), FVIII coding sequence hF8 BDD (438-4811 of SEQ ID NO:9), SPA51 synthetic poly A sequence (nt 4818-4868 of SEQ ID NO:9), and insulator sequence Ins3 (nt 4869-4885 of SEQ ID NO:9), as described in PCT Publication No. WO 2017/074526.

In some embodiments, a method for producing recombinant adeno-associated virus 6 (rAAV6) vector comprising blood clotting Factor VIII is provided. The method comprises (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and ii) culturing the Sf9 cell under suitable conditions and for 108±5 hours to produce rAAV6 vector with no more than 15% clipping between VP1 amino acid residues that correspond to Gly115 and Arg116 of wild-type AAV6.

In some embodiments, a method for producing recombinant adeno-associated virus 6 (rAAV6) vector comprising blood clotting Factor VIII is provided. The method comprises (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and (ii) culturing the Sf9 cell under suitable conditions and for 108±5 hours to produce rAAV6 vector with no more than 65% clipping between VP1 and VP2 amino acid residues that correspond to Gly189 and Glu190 and no more than 15% clipping between VP1 amino acid residues that correspond to Gly115 and Arg116 of wild-type AAV6.

In some embodiments, a method for producing recombinant adeno-associated virus 6 (rAAV6) vector comprising blood clotting Factor VIII is provided. The method comprises (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and (ii) culturing the Sf9 cell under suitable conditions for 108±5 hours to produce rAAV6 vector having an in vitro potency that is between 50-150% as compared to a reference standard with no more than 15% clipping between VP1 amino acid residues that correspond to Gly115 and Arg116 of wild-type AAV6.

In some embodiments, a method for producing recombinant adeno-associated virus 6 (rAAV6) vector comprising blood clotting Factor VIII is provided. The method comprises (i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and (ii) culturing the Sf9 cell under suitable conditions for 108±5 hours to produce rAAV6 vector having an in vitro potency that is between 50-150% as compared to a reference standard with no more than 65% clipping between VP1 and VP2 amino acid residues that correspond to Gly189 and Glu190 and no more than 15% clipping between VP1 amino acid residues that correspond to Gly115 and Arg116 of wild-type AAV6.

In some embodiments, the amount of vector recombinant baculovirus contacted with the insect cell is about 0.01% volume relative to the total culture volume. The amount of helper recombinant baculovirus contacted with the insect cell is about 0.01% volume relative to the total culture volume. The amount of dissolved oxygen in the culture medium is between about 40% to 50% of air saturation. The insect cells are cultured at a temperature of about 26-28° C. for about 108±5 hours prior to recovering the rAAV vector from the insect cell.

Also, disclosed herein are compositions comprising purified, recombinant adeno-associated virus (rAAV) vector. In some embodiments, the compositions comprise rAAV vector with no more than 15% clipping between amino acid residues 115G and 116R on VP1 protein of wild-type AAV6 or the corresponding amino acids in the VP1 and VP2 proteins of another AAV serotype. In some embodiments, the compositions comprise rAAV vector with no more than 15% clipping between amino acid residues 115G and 116R and no more than 65% clipping between amino acid residues 189G and 190E on VP1 and VP2 proteins of wild-type AAV6, or the corresponding amino acids in the VP1 and VP2 proteins of another AAV serotype. The clipping on VP1 and VP2 proteins may be measured by capillary gel electrophoresis (CGE), mass spectrometry (including multi-attribute mass spectrometry), and/or Western blot assays.

As disclosed herein, the rAAV vector may be of any wild type or variant serotype, including but not limited to, rAAV1, rAAV3a, rAAV3b, rAAV6 or rAAV8. In some embodiments, the rAAV vector comprises a transgene that encodes a therapeutic polypeptide, such as but not limited to, wild type or functional variant blood clotting factor, mini-dystrophin, C1 esterase inhibitor, copper transporting P-type ATPase (ATP7B), copper-zinc superoxide dismutase 1 (SOD1) or myosin binding protein C3. In some embodiments, the wild type of functional variant blood clotting factor is Factor VII, Factor VIII or Factor IX. In some embodiments, the wild type or functional variant blood clotting factor is Factor VIII.

In some embodiments, compositions described herein comprise rAAV vector having an in vitro potency of at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard. As described herein, the in vitro potency may be measured using a colorimetric assay, a chromogenic assay or an ELISA-based assay.

In some embodiments, compositions described herein comprise rAAV vector having an in vivo potency of at least 5%, at least 10%, at least 15%, at least 20%, at least 25%, at least 30%, at least 35%, at least 40% at least 45%, at least 50%, at least 55%, at least 60%, at least 65%, at least 70%, at least 75%, at least 80%, at least 85%, at least 90%, at least 95%, at least 100%, at least 105%, at least 110%, at least 115%, at least 120%, at least 125%, at least 130%, at least 135%, at least 140%, at least 145%, at least 150%, at least 160%, at least 170%, at least 180%, at least 190%, or at least 200% as compared to a reference standard. The in vivo potency may be measured in an animal model.

In some aspects, a composition comprising purified, recombinant adeno-associated virus 6 (rAAV6) vector is provided. The rAAV vector comprises a transgene encoding a wild type or functional variant blood clotting factor VIII having an in vitro potency of about 10%-500% as compared to a reference standard with no more than 15% clipping between amino acid residues 115G and 116R on VP1 proteins.

In some aspects, a composition comprising purified, recombinant adeno-associated virus 6 (rAAV6) vector is provided. The rAAV vector comprises a transgene encoding a wild type or functional variant blood clotting factor VIII having an in vitro potency of about 10%-500% as compared to a reference standard with no more than 15% clipping between amino acid residues 115G and 116R and no more than 65% clipping between amino acid residues 189G and 190E on VP1 and VP2 proteins.

In some embodiments, the compositions described herein are pharmaceutical compositions. The pharmaceutical compositions may comprise purified, recombinant adeno-associated virus (rAAV) vector with no more than 15% clipping between amino acid residues 115G and 116R on VP1 proteins, or rAAV vector with no more than 15% clipping between amino acid residues 115G and 116R and no more than 65% clipping between amino acid residues 189G and 190E on VP1 and VP2 proteins of wild-type AAV6, or the corresponding amino acids in the VP1 and VP2 proteins of another AAV serotype, and a pharmaceutically acceptable carrier.

In some embodiments, the pharmaceutical composition comprises a therapeutically effective amount of rAAV6 vector comprising a nucleic acid sequences encoding Factor VIII (e.g., SEQ ID NO: 9), and a pharmaceutically acceptable carrier. The rAAV6 vector has no more than 15% clipping between amino acid residues 115G and 116R on VP1 proteins, or rAAV vector with no more than 15% clipping between amino acid residues 115G and 116R and no more than 65% clipping between amino acid residues 189G and 190E on VP1 and VP2 proteins of wild-type AAV6, or the corresponding amino acids in the VP1 and VP2 proteins of another AAV serotype

In some embodiments, the pharmaceutical compositions described herein further comprises a pharmaceutically-acceptable carrier, adjuvant, diluent, excipient and/or other medicinal agents. A pharmaceutically acceptable carrier, adjuvant, diluent, excipient or other medicinal agent is one that is not biologically or otherwise undesirable, e.g., the material may be administered to a subject without causing undesirable biological effects which outweigh the advantageous biological effects of the material.

Any suitable pharmaceutically acceptable carrier or excipient can be used in the preparation of a pharmaceutical composition according to the invention (See e.g., Remington The Science and Practice of Pharmacy, Alfonso R. Gennaro (Editor) Mack Publishing Company, April 1997).

A pharmaceutical composition is typically sterile, pyrogen-free and stable under the conditions of manufacture and storage. A pharmaceutical composition may be formulated as a solution (e.g., water, saline, dextrose solution, buffered solution, or other pharmaceutically sterile fluid), microemulsion, liposome, or other ordered structure suitable to accommodate a high product (e.g., viral vector particles, microparticles or nanoparticles) concentration. In some embodiments, a pharmaceutical composition comprising rAAV vector of the disclosure is formulated in water or a buffered saline solution. A carrier may be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by use of a coating such as lecithin, by maintenance of a required particle size, in the case of dispersion, and by the use of surfactants. In some embodiments, it may be preferable to include isotonic agents, for example, a sugar, a polyalcohol such as mannitol, sorbitol, or sodium chloride in the composition. Prolonged adsorption of an injectable composition can be brought about by including, in the composition, an agent which delays absorption, e.g., a monostearate salt and gelatin. In some embodiments, a rAAV vector of the disclosure may be administered in a controlled release formulation, for example, in a composition which includes a slow release polymer or other carrier that protects the product against rapid release, including an implant and microencapsulated delivery system.

In some embodiments, a pharmaceutical composition of the disclosure is a parenteral pharmaceutical composition, including a composition suitable for intravenous, intraarterial, subcutaneous, intradermal, intraperitoneal, intramuscular, intraarticular, intraparenchymal (IP), intrathecal (IT), intracerebroventricular (ICV) and/or intracisternal magna (ICM) administration. In some embodiments, a pharmaceutical composition comprising an rAAV vector comprising a modified nucleic acid encoding Factor VIII is formulated for administration by IV injection.

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

### EXAMPLES

The invention is further described in detail by reference to the following examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### Example 1: Relationship Between In Vitro Potency and Batch Duration Post Infection

The rAAV vector, termed PF-07055480 (also called “SB-525” herein) was generated using a baculovirus expression system in Sf9 cells. Cells from a cell bank were expanded to a 2000 L production bioreactor and then co-infected with recombinant baculoviruses carrying rep, cap (helper master baculovirus infected insect cells or MBIIC) and the Factor VIII gene (vector master baculovirus infected insect cells or MBIIC) flanked by inverted terminal repeats. The cell culture process was continued in the production bioreactor for several days until harvest followed by digestion, filtration and purification to generate drug substance.

The drug substance samples were then tested by an in vitro potency assay which includes viral infectivity, gene expression and protein function (FIG. 4). Briefly, liver carcinoma cells were exposed to AAV, allowed to culture for several days, and followed by the use of a chromogenic substrate to quantify Factor VIII activity in harvested media.

The results in FIG. 1 suggest that in vitro potency decreases with batch duration post infection (i.e., after contacting with the recombinant baculoviruses), within the observed range of 103 hours to 163 hours.

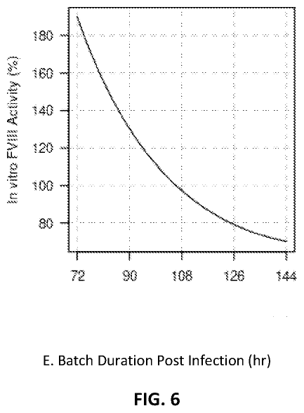

The results were confirmed in small-scale experiments conducted in the 2 L bioreactors, as detailed in FIG. 6 described in Example 3.

### Example 2: Identification of VP1 and VP2 Clipping as Likely Root Cause of Decrease in In Vitro Potency

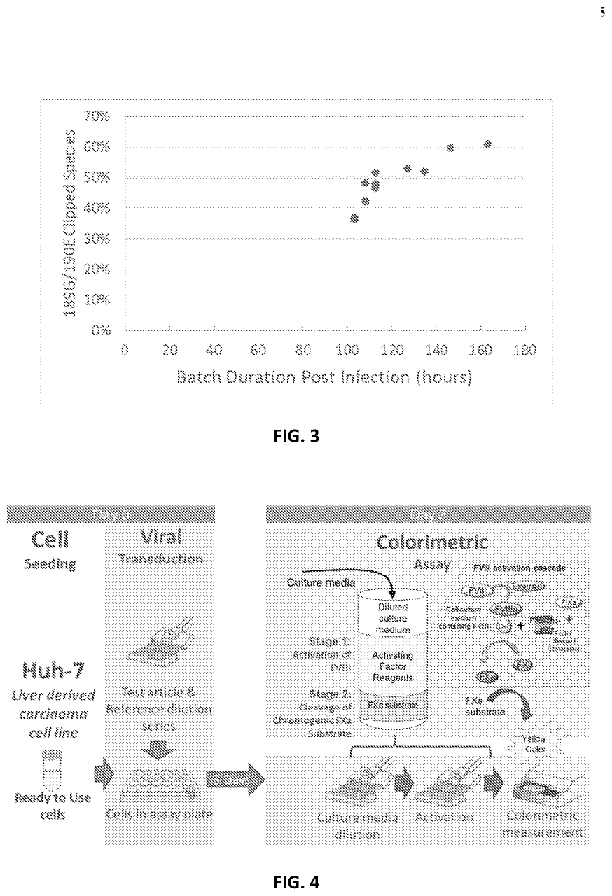

As a result of the variation of observed in vitro potency, additional studies were conducted to understand the potential drivers for the change in in vitro potency values from both the process and analytical perspective. Mass spectrometry (MS) studies identified an attribute that appears to be correlated with in vitro potency. The attribute, a clipped form of the VP1/VP2 protein, was quantified using a LC/MS—peptide mapping method that assesses multiple attributes simultaneously (referred to as multi-attribute method or MAM). This clipped species has been identified as the C-terminal peptide remaining after proteolytic cleavage between amino acid residues ¹⁸⁹G and ¹⁹⁰E on the VP1 and VP2 proteins. The N-terminal peptide from VP1 or VP2 is not detectable and is presumed to be cleared during the manufacturing process. As shown in FIG. 2 and Table 1, the data demonstrate an inverse correlation between the clipped VP1/VP2 protein levels and the relative potency.

TABLE 1
Summary Data of Batch Duration Post Infection and Selected Product
Quality Attributes

| Batch | Batch Duration Post | Clipping @ | Clipping @ | Total | In vitro |
|---|---|---|---|---|---|
| # | Infection (hours) | G115/R116 | G189/E190 | clipping | Potency |
| 1 | 135 | 12.5% | 52.1% | 64.6% | 72% |
| 2 | 163 | 15.4% | 61.0% | 76.4% | 61% |
| 3 | 127 | 12.5% | 53.0% | 65.5% | 76% |
| 4 | 146 | 14.0% | 59.9% | 73.9% | 61% |
| 5 | 103 | 8.3% | 36.3% | 44.6% | 107% |
| 6 | 108 | 12.2% | 48.2% | 60.4% | 82% |
| 7 | 108 | 8.6% | 42.3% | 50.9% | 96% |
| 8 | 113 | 8.9% | 46.8% | 55.7% | 107% |
| 9 | 113 | 10.2% | 51.6% | 61.8% | 82% |
| 10 | 103 | 1.7% | 36.9% | 38.6% | 114% |
| 11 | 113 | 2.5% | 48.0% | 50.5% | 81% |

The data demonstrate an inverse correlation between the clipped VP1/VP2 protein levels and the relative in vitro potency where in vitro potency decreases as the level of clipped VP1/VP2 increases.

### Example 3: Impact of Cell Culture Temperature, Dissolved Oxygen, Amount of Recombinant Baculovirus and Batch Duration Post Infection on In Vitro Potency

A statistically designed experiment was constructed and executed in the 2 L bioreactors to identify influential process parameters in the production of rAAV vector PF-07055480 (also called “SB-525” herein). In particular, impact of process parameters, including cell culture temperature in the production bioreactor, dissolved oxygen level, amount of recombinant baculovirus and batch duration post infection, on rAAV vector attributes including in vitro potency were studied in this experiment.

Briefly, Sf9 cells were serially passaged and expanded to inoculate 2 L production bioreactors. After a target cell density is reached, recombinant baculoviruses preserved in the form of master baculovirus-infected insect cells (MBIIC) are added to initiate AAV production. The culture is continued for a defined period of time, after which it is harvested, clarified, and partially purified. In vitro potency data are collected on the partially purified AAV product.

Based on the experimental data the predictive relationship between these parameters and in vitro potency is summarized in this example.

### Experimental Design and Data Analysis

Central composite design is one of the commonly used designs for response surface modeling that allows for the estimation of the factor interactions and quadratic terms. A minimal fractional factorial with Resolution V design is employed for this study. 6 factors were studied in a total of 60 runs divided among several blocks.

Model building and model reduction was performed considering the software (Design Expert) suggested model, Lack of Fit, Adjusted R², and other model diagnostics, both numerical and visual. Model fitting also included examination of residual plots, actual vs. predicted plots, and consideration of a data transformation. Significance level cutoff for the p-value was chosen to be 0.05.

There is a consistent offset observed in the in vitro potency values measured from partially purified material from the 2 L bioreactors and that of drug substance produced from 2000 L bioreactor, therefore a linear regression model was built to transform the data in the presented model below in order to reflect expected values in drug substance.

Experimental design and data analyses are performed in Design Expert software in version 11.0.6.0.

### In Vitro Potency Results

As shown in FIG. 6, the study confirms the negative relationship between batch duration post infection and potency, as potency decreases as a function of batch duration. A negative relationship is also observed for the ratio of vector MBIIC addition volume relative to culture volume and in vitro potency. A positive relationship is observed for the ratio of helper MBIIC addition volume relative to culture volume and in vitro potency. A quadratic effect of temperature is observed, whereas the optimal temperature for potency is between 27-28 C, and potency decreases with temperature deviating from the optimum. A positive relationship is observed for dissolved oxygen and in vitro potency.

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

What is claimed:

1. A method for increasing in vitro potency of a recombinant adeno-associated viral (AAV) vector comprising:
contacting an insect cell with one or more recombinant baculovirus(es), each baculovirus comprising a heterologous sequence; and
optimizing the time for culturing the insect cell under suitable conditions such that the in vitro potency of the rAAV vector is between 10%-500% as compared to a reference standard with no more than 65% clipping between VP1 and VP2 amino acid residues that correspond to Gly189 and Glu190 and no more than 15% clipping between VP1 amino acid residues that correspond to Gly115 and Arg116 of wild-type AAV6, or the corresponding amino acids in the VP1 and VP2 proteins of another AAV serotype.

2. The method of claim 1, wherein the rAAV vector has between about 30% and about 60% clipping between VP1 and VP2 amino acid residues that correspond to Gly189 and Glu190 and no more than 5% clipping between VP1 amino acid residues that correspond to Gly115 and Arg116 of wild-type AAV6, or the corresponding amino acids in the VP1 and VP2 proteins of another AAV serotype.

3. The method of claim 1, wherein the clipping on VP1 and VP2 proteins is measured by capillary gel electrophoresis (CGE), mass spectrometry (including multi-attribute mass spectrometry), and/or Western blot assays.

4. The method of claim 1, wherein the in vitro potency of the rAAV vector is between 50% and 150% as compared to a reference standard.

5. The method of claim 1, wherein the in vitro potency is measured using a colorimetric assay, a chromogenic assay, an ELISA-based assay, quantitative PCR, and/or Western blot.

6. The method of claim 1, wherein the insect cell is cultured for about 96 hours to about 128 hours prior to recovering the rAAV vector from the insect cell, or for about 108±5 hours prior to recovering the rAAV vector from the insect cell.

7. The method of claim 1, wherein the insect cell is contacted with:
(i) one or two helper recombinant baculovirus(es), each baculovirus comprising a heterologous sequence encoding AAV Rep proteins and/or AAV Cap proteins, and
(iii) a vector recombinant baculovirus comprising a heterologous sequence encoding a transgene between two AAV inverted terminal repeats (ITRs).

8. The method of claim 7, wherein suitable conditions for culturing the insect cell that produce rAAV vector with no more than 65% clipping between amino acid residues 189G and 190E and no more than 15% clipping between amino acid residues 115G and 116R on VP1 proteins of wild-type AAV6, or the corresponding amino acids in the VP1 and VP2 proteins of another AAV serotype, comprise:
(i) temperature at which the insect cell is cultured,
(ii) amount of helper recombinant baculovirus contacted with the insect cell,
(iii) amount of vector recombinant baculovirus vector contacted with the insect cell, and/or
(iv) amount of dissolved oxygen in the cell culture medium.

9. The method of claim 8, wherein the insect cell is cultured at a temperature of about 25° C., about 26° C., about 27° C., about 28° C., about 29° C., about 30° C. or about 31° C.

10. The method of claim 8, wherein the amount of helper recombinant baculovirus contacted with the insect cell is between about 0.0022% to about 0.0178% volume relative to the total culture volume.

11. The method of claim 8, wherein the amount of vector recombinant baculovirus contacted with the insect cell is between about 0.0022% to about 0.0178% volume relative to the total culture volume.

12. The method of claim 8, wherein the amount of dissolved oxygen in the culture medium is about 20% to about 100% of air saturation.

13. The method of claim 1, wherein the insect cell is Sf9 cell, Sf21 cell or Hi5 cell.

14. The method of claim 7, wherein the transgene encodes a wild type or functional variant blood clotting factor, mini-dystrophin, C1 esterase inhibitor, copper transporting P-type ATPase (ATP7B), copper-zinc superoxide dismutase 1 (SOD1) or myosin binding protein C3.

15. The method of claim 14, wherein the wild type of functional variant blood clotting factor is Factor VII, Factor VIII or Factor IX.

16. The method of claim 1, wherein the rAAV is rAAV1, rAAV3a, rAAV3b, rAAV6, or rAAV8.

17. A method for producing recombinant adeno-associated virus 6 (rAAV6) vector comprising blood clotting Factor VIII, the method comprising:
(i) contacting a Sf9 cell with one or two helper recombinant baculovirus(es), each helper recombinant baculovirus comprising a heterologous sequence encoding AAV6 Rep proteins and/or AAV6 Cap proteins, and a vector recombinant baculovirus comprising a heterologous sequence encoding blood clotting Factor VIII between two AAV2 inverted terminal repeats (ITRs); and
(ii) culturing the Sf9 cell under suitable conditions for 108±5 hours to produce rAAV6 vector having an in vitro potency that is between 50-150% as compared to a reference standard with no more than 65% clipping between VP1 and VP2 amino acid residues that correspond to Gly189 and Glu190 and no more than 15% clipping between VP1 amino acid residues that correspond to Gly115 and Arg116 of wild-type AAV6.

18. The method of claim 17, wherein
(i) the insect cell is cultured at a temperature of about 28° C.,
(ii) the amount of vector recombinant baculovirus contacted with the insect cell is between about 0.0022% to about 0.0178% volume relative to the total culture volume,
(iii) the amount of helper recombinant baculovirus contacted with the insect cell is between about 0.0022% to about 0.0178% volume relative to the total culture volume, and
(iv) the amount of dissolved oxygen in the culture medium is about 20% to about 100% of air saturation.

19. A composition comprising purified, recombinant adeno-associated virus (rAAV) vector with no more than 15% clipping between amino acid residues 115G and 116R and no more than 65% clipping between amino acid residues 189G and 190E on VP1 and VP2 proteins of wild-type AAV6 or the corresponding amino acids in the VP1 and VP2 proteins of another AAV serotype.

20. A composition comprising purified, recombinant adeno-associated virus 6 (rAAV6) vector comprising a transgene encoding a wild type or functional variant blood clotting factor VIII having an in vitro potency of about 50%-150% as compared to a reference standard with no more than 15% clipping between amino acid residues 115G and 116R and no more than 65% clipping between amino acid residues 189G and 190E on VP1 and VP2 proteins.


SEQUENCE LISTING (16 sequences)

SEQ ID NO: 1 - protein, 736 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, Artificial Sequence
  Synthetic Construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 3 - protein, 736 aa, Artificial Sequence
  Synthetic Construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 736 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - protein, 738 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - protein, 7 aa, Artificial Sequence
  Synthetic Construct
QAKKRVL

SEQ ID NO: 7 - protein, 7 aa, Artificial Sequence
  Synthetic Construct
PGKKRPV

SEQ ID NO: 8 - protein, 7 aa, Artificial Sequence
  Synthetic Construct
PARKRLN

SEQ ID NO: 9 - DNA, 4894 bp, Artificial Sequence
  Synthetic Construct
gcggcctaagcttggaaccattgccaccttcagggggaggctgctggtgaatattaaccaagatcaccccagttaccggaggagcaaacagggactaagttcacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagttatcgttagttcgtgcaccattaatgtttaattacctggagcacctgcctgaaatcatttttttttcaggttggctagtatgcagatcgagctctccacctgcttctttctgtgcctgttgagattctgcttcagcgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatccacccgtccttaagcgccatcagcgcgagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcatctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaggatccaataaaatatctttattttcattacatctgtgtgttggttttttgtgtgttttcctgtaacgatcgggctcgagcgc

SEQ ID NO: 10 - DNA, 130 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 108 bp, Artificial Sequence
  Synthetic Construct
aggaacccctagtgatggagttggccactccctctctgcgcgctcgctcgctcactgaggccgcccgggctttgcccgggcggcctcagtgagcgagcgagcgcgcag

SEQ ID NO: 12 - protein, 1457 aa, Artificial Sequence
  Synthetic Construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - protein, 19 aa, Artificial Sequence
  Synthetic Construct
MQIELSTCFFLCLLRFCFS

SEQ ID NO: 14 - DNA, 84 bp, Artificial Sequence
  Synthetic Construct
gggggaggctgctggtgaatattaaccaagatcaccccagttaccggaggagcaaacagggactaagttcacacgcgtggtacc

SEQ ID NO: 15 - DNA, 223 bp, Artificial Sequence
  Synthetic Construct
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 16 - DNA, 4374 bp, Artificial Sequence
  Synthetic Construct
atgcagatcgagctctccacctgcttctttctgtgcctgttgagattctgcttcagcgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatccacccgtccttaagcgccatcagcgcgagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcatctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga